(19) United States
(12) Patent Application Publication
(10) Pub. No.: US 2006/0123509 A1
(43) Pub. Date: Jun. 8, 2006

(54) Protein isolation and purification

(76) Inventors: Margarita Torrent, Barcelone (ES); Miriam Bastida, Molins de Rei (ES); Pablo Marzabal, Barcelone (ES); Blanca Llompart, Barcelone (ES); Ma Dolores Ludevid, Sant Just Desvern (ES)

Correspondence Address: WELSH & KATZ, LTD, 120 S RIVERSIDE PLAZA, 22ND FLOOR, CHICAGO, IL, 60606, US

(21) Appl. No.: 11/289,264

(22) Filed: Nov. 29, 2005

(30) Foreign Application Priority Data
Nov. 29, 2004 (GB) 0426161.6

(51) Int. Cl. A01H 1/00; C12P 21/06; C07K 14/425; C12N 1/18

(52) U.S. Cl. 800/278; 435/69.1; 435/325; 435/320.1; 435/254.1; 530/370

(57) ABSTRACT

A method for purifying a recombinant fusion protein that is expressed as recombinant protein body-like assemblies (RPBLAs) in host cells is disclosed in which an aqueous homogenate of transformed host cells that express a fusion protein as RPBLAs having a predetermined density is provided. Regions of different density are formed in the homogenate to provide a region that contains a relatively enhanced concentration of the RPBLAs and a region that contains a relatively depleted concentration of the RPBLAs. The RPBLAs-depleted region is separated from the region of relatively enhanced concentration of RPBLAs, thereby purifying said fusion protein. The region of relatively enhanced concentration of RPBLAs can thereafter be collected as desired.

### TECHNICAL FIELD

The present invention provides a method for purifying recombinant proteins accumulated in recombinat protein bodies-like assemblies (RPBLAs). More specifically, the invention provides for the isolation of recombinant fusion proteins within recombinant protein bodies-like assemblies that permit isolation from other host-cell organelles by a difference in density wherein the desired recombinant protein can be concentrated, separated from other cell components and easily recovered.

### BACKGROUND ART

Protein bodies (PBs) are subcellular organelles (or large vesicles, about 1-3 microns in diameter, surrounded by a membrane) that specialize in protein accumulation. They are naturally formed in some specific plant tissues, like seeds, and serve as principal source of amino acids for germination and seedling growth.

The storage proteins are co-translationally inserted into the lumen of the endoplasmic reticulum (ER) via a signal peptide to be packaged either in the ER or into the vacuoles (Galili et al., 1993 Trends Cell Biol. 3:437-443) and assembled into multimeric units inside these subcellular compartments, developing specific organelles called (ER)-derived protein bodies (PBs) or protein storage vacuoles (PSV) (Okita and Rogers, 1996 Annu. Rev. Plant Physiol Mol. Biol. 47:327-350; Herman and Larkins, 1999 Plant Cell 11:601-613; Sanderfoot and Raikel, 1999 Plant Cell 11:629-642).

The storage proteins dicotiledoneous plants are primarily soluble proteins such as the 7S globulin or vicilin type, 11S globulins or legumin-type proteins and are sequestered in PSVs together with other proteins (i.e., protease inhibitors, proteolytic enzymes, lectins and the like), sugars and salts.

In contrast to PSVs, PBs (1-3 microns) sequester predominantly prolamins, which are highly hydrophobic storage proteins of cereals (such as zeins of maize and gliadins of wheat), and lack of other auxiliary proteins (Herman and Larkins, 1999 Plant Cell 11:601-613).

At present, no PBs have been found in tissues other than plant seeds, with the exception of the ER bodies. The ER bodies are small in size (0.2-0.4 micrometers) and are formed in Arabidopsis leaves only by wounding and chewing by insects but do not develop under normal conditions (Matsushima et al., 2003 Plant J. 33:493-502).

Genetic engineering approaches have been used to study plant PBs formation, storage protein assembly and targeting. It has been shown that when recombinant proteins, predominantly plant storage proteins are expressed and packaged in Arabidopsis and tobacco, plant tissues that did not contain PBs (as vegetative tissues), develop these organelles “de novo” (Bagga et al., 1997 Plant Cell 9:1683-1696 and Bagga et al., 1995 Plant Physiol. 107:13-23, and U.S. Pat. No. 5,990,384, U.S. Pat. No. 5,215,912, and U.S. Pat. No. 5,589,616; and Geli et al., 1994 Plant Cell 6:1911-1922).

Maize beta-zein when expressed in transgenic tobacco plants was correctly targeted in new formed ER-derived PBs in leaf cells (Bagga et al., 1995 Plant Physiol. 107:13-23). Maize gamma-zein and, truncated gamma-zein cDNAs expressed in Arabidopsis plants also accumulate in a novel ER-derived PBs in leaves (Geli et al., 1994 Plant Cell 6:1911-1922). Lysine-rich gamma-zeins expressed in maize endosperms (Torrent et al. 1997 Plant Mol. Biol. 34(1):139-149) accumulate in maize PBs and co-localized with endogenous zeins. Transgenic tobacco plants expressing alpha-zein gene demonstrated that alpha-zein was not able to form PBs. However, when alpha- and gamma-zein were co-expressed, the stability of alpha-zein increased and both proteins co-localized in ER-derived protein bodies (Coleman et al., 1996 Plant Cell 8:2335-2345). Formation of novel PBs has been also described in transgenic soybean transformed with methionine-rich 10 kDa delta-zein (Bagga et al., 2000 Plant Sci. 150:21-28).

Recombinant storage proteins are also assembled in PBs-like organelles in a non-plant host system such as Xenopus oocytes and in yeast. Rosenberg et al., 1993 Plant Physiol 102:61-69 reported the expression of wheat gamma-gliadin in yeast. The gene expressed correctly and the protein was accumulated in ER-derived PBs. In Xenopus oocytes, Torrent et al., 1994 Planta 192:512-518 demonstrated that gamma zein also accumulates in PB-like organelles when transcripts encoding the protein were microinjected into oocytes. Hurkman et al., 1981 J. Cell Biol. 87:292-299 with alfa-zeins and Altschuler et al., 1993 Plant Cell 5:443-450 with gamma-gliadins had similar results in Xenopus oocytes.

One of the fundamental achievements of the field of the biotechnology (genetic engineering) is the ability to genetically manipulate an organism to produce a protein for therapeutic, nutraceutical or industrial uses. Methods are provided for producing and recovering recombinant proteins from fermentation broth of bacteria, yeast, crop plants and mammalian cell cultures. Different approaches for protein expression in host cells have been described. The essential objectives of these approaches are: protein expression level, protein stability and protein recovery (Menkhaus et al., 2004 Biotechnol. Prog. 20: 1001-1014; Evangelista et al., 1998 Biotechnol. Prog. 14:607-614).

One strategy that can solve a problem with protein recovery is secretion. However, secretion involves some times poor expression levels and product instability. Another strategy is the accumulation of the recombinant protein in the most beneficial location in the cell. This strategy has been extensively used by directing recombinant proteins to the ER by engineering C-terminal extension of a tetrapeptide (HDEL/KDEL) (Conrad and Fiedler, 1998 Plant Mol. Biol. 38:101-109).

Fusion proteins containing a plant storage protein or storage protein domains fused to the heterologous protein have been an alternative approach to direct recombinant proteins to the ER (WO 2004003207). One interesting fusion strategy is the production of recombinant proteins fused to oleosins, constitutive protein of plant oil bodies. The specific characteristics of oil bodies benefit of the easy recovery of proteins using a two-phase system (van Rooijen and Moloney, 1995 Bio/Technology 13:72-77).

Heterologous proteins have been successfully expressed in plant cells (reviews Horn et al., 2004 Plant Cell Rep. 22:711-720; Twyman et al., 2003, Trends in Biotechnology 21:570-578; Ma et al., 1995, Science 268: 716-719; Richter et al., 2000 Nat. Biotechnol. 18:1167-1171), and in some, the expression of the recombinant protein has been directed to ER-derived PB or PSV (PSV). Yang et al., 2003 Planta 216:597-603, expressed human lysozyme in rice seeds using the seed-specific promoters of glutelin and globulin storage proteins. Immunocytochemistry results indicated that the recombinant protein was located in ER-PBs and accumulated with endogenous rice globulins and glutelins. The expression of glycoprotein B of the human cytomegalovirus (hCMV) in transgenic tobacco plants has been carried out using a glutelin promoter of rice. Tackaberry et al., 1999 Vaccine 17:3020-3029. Recently, Arcalis et al., 2004 Plant Physiology 136:1-10 expressed human serum albumin (HSA) with a C-terminal extension (KDEL) in rice seeds. The recombinant HSA accumulated in PSVs with the endogenous rice storage proteins.

One obstacle for the application of plants as biofactories is the need for more research regarding the downstream processing. Protein purification from plants is a difficult task due to the complexity of the plant system. Plant solids of the extract are large, dense and relative elevated (9-20 percent by weight) (see review Menkhaus et al., 2004 Biotechnol. Prog. 20:1001-1014). At present, recombinant protein purification techniques include clarification of the extracts, treatment with solvents to remove lipids and pigments and protein or peptides purification by several ion-exchange and gel-filtration chromatography columns. The existing protocols rely upon the use of specific solvents or aqueous solutions for each plant-host system and recombinant protein. There is a need in the art for efficient and general procedures for recombinant protein recovery from transformed hosts. This need is especially relevant in cases where recombinant proteins produced in plant hosts must to be isolated. The diversity of hosts and proteins and the different physical-chemical traits between them required an efficient method to concentrate and recover recombinant products. The invention disclosed hereinafter provides one means for easing and enhancing the recovery of recombinantly-expressed proteins from non-higher plant organisms such as fungi and mammalian cells.

### BRIEF SUMMARY OF THE INVENTION

The present invention provides an efficient and general procedure or method for recombinant protein recovery from transformed hosts. The solution presented herein is based on the discovery that the isolation of the recombinat protein bodies-like assemblies (RPBLAs) from other host-cell proteins can be effected with unexpectedly good yields by a density-based technique, in particular by density cushion or density gradient centrifugation techniques.

More particularly, the present invention contemplates a method for purifying a recombinant fusion protein that is expressed as RPBLAs in host cells. In accordance with a contemplated method, a aqueous homogenate of transformed host cells that express a fusion protein as RPBLAs is provided. Those RPBLAs have a predetermined density that can differ among different fusion proteins, but is known for a particular fusion protein to be separated. That predetermined density of the RPBLAs is typically greater than that of substantially all of the endogenous host cell proteins present in the homogenate, and is typically about 1.1 to about 1.35 g/ml. Regions of different density are formed in the homogenate to provide a region that contains a relatively enhanced concentration of the RPBLAs and a region that contains a relatively depleted concentration of the RPBLAs. The RPBLAs-depleted region is separated from the region of relatively enhanced concentration of RPBLAs, thereby purifying said fusion protein. The region of relatively enhanced concentration of RPBLAs can thereafter be collected or can be treated with one or more reagents or subjected to one or more procedures prior to isolation of the RPBLAs or the fusion protein therein.

In preferred practice, the RPBLAs contains two polypeptide sequences linked together in which one sequence is that of a protein body-inducing sequence (PBIS) whereas the other is the sequence of a product of interest such as a drug molecule, and enzyme or the like. Preferred protein body-inducing sequences are those of prolamin compounds such as gamma-zein, alpha-zein or rice prolamin.

The host cells here are eukaryotic cells such as those of higher plants, yeasts and fungi, animal cells such as mammalian cells and algal cells. Those cells can be fresh as are obtained directly from fresh biomass, or can be dried as are obtained from dried biomass. Biomass is the mass obtained from living organisms or cells, such as a culture medium or a living organism as, for instance, a plant leaf.

### BRIEF DESCRIPTION OF THE DRAWINGS

In the drawings forming a part of this disclosure,

FIG. 1 is s schematic representation of the binary vectors used in the transient (agroinfiltration) and stable transformation of tobacco plants are shown at the top of the figure. The two vectors used in yeast transformation are represented in the middle of the figure. The vectors used in the transient transfection of mammalian cell cultures are indicated at the bottom. RX3, N terminal domain of 27 kD gamma-zein, 22aZ, alpha-zein of 22 kD, 22aZt, N-terminal domain of the alpha-zein of 22 kD, rP13, 13 kD rice prolamin and CS, cleavage site.

FIG. 2 is in four parts, FIGS. 2A-2D. FIG. 2A shows RX3-T20 and RX3-EGF fusion proteins accumulation in leaves of transgenic tobacco plants. Soluble proteins were extracted from wild type (wt) and transgenic tobacco leaves (lanes 2 and 4), analyzed on SDS-polyacrylamide gels, and transferred to nitrocellulose membranes followed by immunoblot analysis using gamma-zein antiserum. Molecular weights are indicated on the left. RX3-derived fusions monomer (M) and dimers (D) are indicated by arrows.

FIG. 2B shows the immunoblot analysis of RX3-T20 and RX3-EGF fusion proteins in density gradient fractions. Clarified homogenates of wet (fresh) leaves of transformed tobacco were loaded on a step sucrose gradient (42%-49%-56%-65% w/w). RX3-EGF and RX3-T20 fusion proteins accumulation in the homogenate, supernatant, interphase and pellet fractions were analyzed by immunoblot using gamma-zein antibody. Each lane corresponds to equivalent volumes of all fractions. H, homogenate; S, supernatant; F42, interphase 42-49% w/w; F49, interphase 49-56% w/w; F56, interphase 56-65% w/w; F65, pellet under 65% sucrose. Molecular weights are indicated on the left. RX3-derived fusions monomer (M) and dimers (D) are indicated by arrows.

FIG. 2C shows the SDS-PAGE and silver stain analysis of RX3-EGF fusion protein expressed by p19RX3EGF in transformed tobacco leaves in density gradient fractions. Clarified homogenates of wet (fresh) leaves of tobacco in buffer PBP were loaded on a step sucrose (42%-49%-56%-65% w/w) gradient. RX3-EGF fusion protein accumulation in the homogenate, supernatant, interphase and pellet fractions were analyzed by 15% SDS-PAGE and developed by silver stain. Each lane corresponds to equivalent volumes of all fractions. Arrowheads indicate the RX3-EGF protein. H, homogenate; S, supernatant; F42, interphase 42-49% w/w sucrose; F49, interphase 49-56% w/w; F56, interphase 56-65% w/w; F65, pellet under 65% sucrose. Molecular weights are indicated on the left.

FIG. 2D shows SDS-PAGE and immuno-blot results of RX3-T20 and RX3-EGF accumulation in wet and dry tobacco leaves. Tobacco leaves were dried at 37° for one week and stored for five months in a humidity-free container. Soluble proteins extracted from equivalent amounts of wet (W) and dried (D) transformed tobacco leaves were analyzed by SDS-polyacrylamide gels and immunoblot using gamma-zein antiserum (lanes 1 and 2). The accumulation of RX3-EGF and RX3-T20 in dense structures in dried samples was analyzed by fractionation of dry leaf homogenates in sucrose gradients (20%-30%-42%-56% w/w). RX3-EGF and RX3-T20 fusion proteins accumulation in the supernatant, interphase and pellet fractions was analyzed by immunoblot using gamma-zein antibody. Equivalent amounts of each fraction were loaded. S, supernatant; F20, interphase 20%-30% w/w sucrose; F30, interphase 30%-42% w/w sucrose; F42, interphase 42%-56% w/w sucrose; F56, pellet under 56% sucrose.

FIG. 3 is in two parts, FIGS. 3A and 3B. FIG. 3A shows RX3-EGF and RX3-T20 accumulation in agroinfiltrated tobacco plantlets. Total soluble proteins were analyzed by SDS-PAGE and immunoblot using gamma-zein antiserum. Wt=wild type control tobacco plantlets; molecular weights are indicated on the left. RX3-derived fusions monomer (M), dimers (D) and trimers (T) are indicated by arrows.

FIG. 3B shows subcellular fractionation of agroinfiltrated tobacco plantlets. Clarified plantlet homogenates were loaded on step sucrose (20%-30%-42%-56% w/w) gradients. RX3-T20 and RX3-hGH fusion proteins accumulation in the supernatant, interphase and pellet fractions was analyzed by immunoblot using gamma-zein antibody. Equivalent amounts of each fraction were loaded per lane. S, supernatant; F20, interphase 20%-30% w/w sucrose; F30, interphase 30%-42% w/w sucrose; F42, interphase 42%-56% w/w sucrose; F56, pellet under 56% sucrose. Molecular weights are indicated on the left. RX3-derived fusions monomer (M) and dimers (D) are indicated by arrows.

FIG. 4 is in three parts as FIGS. 4A, 4B and 4C. FIG. 4A shows RX3-T20 and RX3-EGF protein concentration after centrifugation through a sucrose cushion. Clarified homogenates of transgenic tobacco leaves were loaded on a sucrose cushion (42% w/w). After centrifugation, RX3-EGF and RX3-T20 fusion proteins accumulation in the supernatant and pellet were analyzed by immunoblot using gamma-zein antibody. Each lane corresponds to equivalent amounts of the fractions. H, homogenate; S, supernatant; P, pellet cushion. Molecular weights are indicated on the left. RX3-derived fusions monomer (M) and dimers (D) are indicated by arrows.

FIG. 4B shows RX3-EGF protein purification after centrifugation through a sucrose cushion. Clarified homogenates of transgenic tobacco leaves were loaded on a sucrose cushion (42% w/w). After centrifugation, protein patterns of the homogenate, supernatant and pellet fractions were analyzed by 15% SDS-PAGE and silver stain. Each lane corresponds to equivalent amounts of the fractions. RX3-EGF monomer (M) and dimers (D) are indicated by arrows. H, homogenate; S, supernatant; P, pellet cushion. Molecular weights are indicated on the left.

FIG. 4C shows RX3-EGF protein concentration and purification after low speed centrifugation (LSC). Clarified homogenates of RX3-EGF-expressing tobacco leaves were centrifuged at 1000×g for 10 minutes and pellet (P1, lane 2) and supernatant (S, lane 1) were analyzed by gel electrophoresis and immunoblot using gamma-zein antibody. The low speed centrifugation (LSC) pellet P1 was washed in a buffered 5% TritonX-100-containing medium and after a second centrifugation, equivalent amounts of the LSC P1 pellet (lane 7), the supernatant after washing (W, lane 8) and the final pellet P2 (lane 9) were analyzed by 15% SDS-PAGE and silver stain. These samples were compared with the equivalent samples (lanes 3-5) from the pellet P1 (lane 3) obtained after one sucrose cushion centrifugation and submitted to the same washing procedure as the LSC pellet. RX3-EGF monomer (M) and dimers (D) are indicated by arrows.

FIG. 5 is in two parts as FIGS. 5A and 5B. FIG. 5A shows subcellular distribution of RX3-Ct, RX3-EGF and RX3-hGH recombinant fusion proteins accumulated in transfected mammal cells. Transfected cell homogenates were loaded on step sucrose (20%-30%-42%-56% w/w) gradients. After centrifugation RX3-Ct, RX3-EGF and RX3-hGH fusion proteins accumulation in the supernatant, interphase and pellet fractions was analyzed by immunoblot using gamma-zein antibody. Cells transfected with plasmid pECFP-N1 (Clontech) expressing ECGP, a cyan fluorescent variant of GFP, were used as a control and ECGP was immunodetected by using an anti-GFP antiserum. H, homogenate; S, supernatant; F20, interphase 20%-30% w/w sucrose; F30, interphase 30%-42% w/w sucrose; F42, interphase 42%-56% w/w sucrose; F56, pellet under 56% sucrose.

FIG. 5B shows CHO-expressed RX3-EGF protein concentration after low speed centrifugation. Homogenates from RX3-EGF-expressing CHO cells were centrifuged at 2500×g for 10 minutes and pellet (P, lane 2) and supernatant (S, lane 1) were analyzed by gel electrophoresis and immunoblot using gamma-zein antibody. Molecular weights are indicated on the left.

FIG. 6 shows subcellular distribution of RX3-EGF and RX3-hGH recombinant fusion proteins accumulated in transformed yeast cells. Lysed spheroplasts from transformed yeast were loaded on step sucrose (20%-30%-42%-56% w/w) gradients. After centrifugation, RX3-EGF and RX3-hGH fusion proteins accumulation in the supernatant, interphase and pellet fractions was analyzed by immunoblot using gamma-zein antibody. H, lysed spheroplasts homogenate; S, supernatant; F20, interphase 20%-30% w/w sucrose; F30, interphase 30%-42% w/w sucrose; F42, interphase 42%-56% w/w sucrose; F56, pellet under 56% sucrose. Molecular weights are indicated on the left.

FIG. 7A shows subcellular distribution of recombinant fusion proteins accumulated in agroinfiltrated tobacco plantlets. Clarified plantlet homogenates were loaded on step sucrose (20%-30%-42%-56% w/w) gradients. The rP13-Ct, rP13-EGF and rP13-hGH fusion protein accumulation in the supernatant, interphase and pellet gradient fractions was analyzed by immunoblot using anti-calcitonin, anti-EGF and anti-hGH antibodies. An equivalent study was performed using two versions of the alpha-zein gene. Calcitonin (Ct) and EGF were fused to the N-terminal domain of the alpha zein (22aZt) and the hGH was fused to a complete alpha zein gene (22aZ). Equivalent amounts of each fraction were loaded per lane. S, supernatant; F20, interphase 20%-30% w/w sucrose; F30, interphase 30%-42% w/w sucrose; F42, interphase 42%-56% w/w sucrose; F56, pellet under 56% sucrose.

FIG. 7B shows results using clarified leaf homogenates from transgenic tobacco lines loaded on step sucrose (10%-42%-56%-62% w/w) gradients. The immunoblot using anti-EGF antibody shows the distribution of the rP13-EGF and 22aZt-EGF of equivalent amounts of each fraction. S, supernatant; F10, interphase 10%-42% w/w sucrose; F42, interphase 42%-56% w/w sucrose; F56, interphase 56%-62% w/w sucrose; F62, pellet under 62% sucrose.

FIG. 8 shows RX3-T20 and RX3-EGF fusion protein recovery from RPBLAs. RPBLA fractions obtained from a density cushion or step gradient were resuspended in the presence of reducing agents. Solubilized (S) and non-solubilized proteins (P) were analyzed by immunoblot using gamma-zein antiserum. Molecular weights are indicated on the left. RX3-derived fusions monomer (M), dimers (D) and trimers (T) are indicated by arrows.

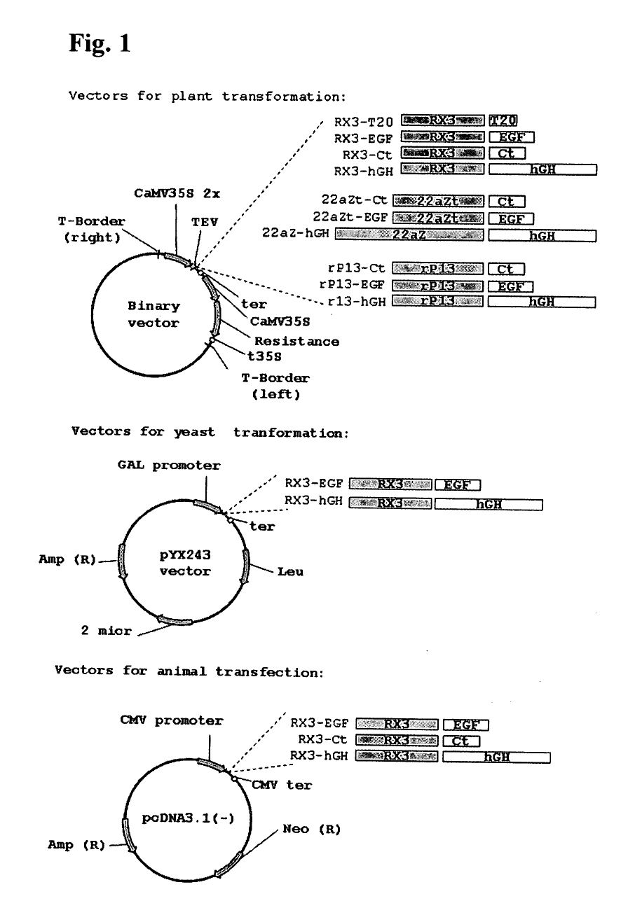

The present invention has several benefits and advantages.

One benefit is that its use enables relatively simple and rapid purification of expressed proteins based on differences in density of the expressed product from the remainder of the soluble cellular materials.

Thus, an advantage of the invention is that it provides a method to eliminate endogenous compounds (or non-recombinant products) from the host-organism and cell cultures.

Another benefit of the invention is that it provides a reliable and reproducible way to purify recombinant peptides or proteins from fresh or dried biomass (host-organism).

### DETAILED DESCRIPTION OF PREFERRED EMBODIMENTS

The present invention relates generally to a downstream process for isolating and purifying recombinant proteins and peptides of interest from transformed organisms or cell cultures. More particularly, the present invention contemplates a method for purifying a recombinant fusion protein that is expressed and accumulates as recombinant protein body-like assemblies (RPBLAs) in host cells. The RPBLAs are recombinant fusion protein assemblies induced by storage protein domains that form high density deposits inside the cells. These dense deposits can accumulate in the cytosol in the endomembrane system organelles, mitochondria, plastids or can be secreted. In accordance with a contemplated method, an aqueous homogenate of transformed host cells that express a fusion protein as RPBLAs is provided. The homogenate is preferably clarified for use. Regions of different density are formed in the homogenate to provide a region that contains a relatively enhanced concentration of the RPBLAs and a region that contains a relatively depleted concentration of the RPBLAs. The RPBLA-depleted region is separated from the region of relatively enhanced concentration of RPBLAs, thereby purifying the fusion protein. The region of relatively enhanced concentration of RPBLAs can thereafter be collected or can be treated with one or more reagents or subjected to one or more procedures prior to isolation of the RPBLAs or the fusion protein therein.

In preferred practice, the fusion protein contains two polypeptide sequences linked together in which one sequence is that of a protein body-inducing sequence (PBIS), whereas the other is the sequence of a polypeptide product of interest such as a drug molecule, and enzyme or the like. Preferred PBIS are those of prolamin compounds such as gamma-zein, alpha-zein or rice prolamin.

One aspect of the present method comprises the provision of an aqueous homogenate or other appropriate extract (collectively referred to herein as a homogenate) of a host-organism or cell culture that expresses and accumulates the desired fusion protein as recombinant protein body-like assemblies (RPBLAs). The homogenate is typically pre-clarified (clarified) prior to use to remove cellular debris as by filtration. The homogenate containing fusion protein-containing protein body-like structures (RPBLAs), lipids, soluble proteins, cell organelles, sugars, pigments and alkaloids is directly loaded on a step density gradient and the homogenate is separated on the basis of the density of its constituents, as by centrifugation. Regions of different density are formed in the homogenate during the centrifugation to provide a region that contains a relatively enhanced concentration of the RPBLA and a region that contains a relatively depleted concentration of the RPBLA. The desired fusion protein-containing RPBLAs can be collected at a specific density interphase. This procedure has permitted the recovery of more than about 90 percent of the expressed recombinant fusion protein at a more than about 80 percent purity.

Another aspect of the invention contemplates a method for RPBLA isolation from a preferably clarified homogenate by one-step density cushion. Here, a preferably clarified homogenate is loaded on a specific density cushion so that endogenous-contaminant compounds do not cross the density cushion and centrifugally separated so that the dense RPBLAs cross the cushion and can be collected. The before-discussed regions of different density are formed in the homogenate to provide a region that contains a relatively enhanced concentration of the RPBLA (the region below the cushion) and a region that contains a relatively depleted concentration of the RPBLA (the region above the cushion). Thus, the density of the recombinant protein body-like assemblies is greater than that of the cushion.

In yet another embodiment, the preferably clarified homogenate is centrifugally separated directly and in the absence of a sucrose or other added density-providing solute. Again, the centrifugation provides regions of different density are formed in the homogenate to provide a region that contains a relatively enhanced concentration of the RPBLA (pellet) and a region that contains a relatively depleted concentration of the RPBLA (supernatant). The RPBLAs can thereafter be separated, to provide purification of the RPBLAs and thereby the fusion protein.

The invention provides a method for recovery of recombinant peptides or protein expressed within RPBLAs, organelles formed in transformed host cells. The host cells here are eukaryotic cells such as those of higher plants, yeasts and fungi, animal cells such as cultured mammalian cells, cells from transgenic animals, animal eggs and the like, and algal cells. Those cells can be fresh as are obtained directly from a culture medium or living organism such as a plant leaf or animal, or can be dried.

The recombinant protein body-like assemblies have a predetermined density that can differ among different fusion proteins, but is known for a particular fusion protein to be separated. That predetermined density of the RPBLAs is typically greater than that of substantially all of the endogenous host cell proteins present in the homogenate, and is typically about 1.1 to about 1.35 g/ml. The high density of novel RPBLAs is due to the general ability of the recombinant fusion proteins to assemble as multimers and accumulate.

The contemplated RPBLAs are expressed in eukaryotes and are typically characterized by their densities as noted above. When expressed in higher plant and animal cells, the RPBLAs are typically spherical in shape, have diameters of about 1 micron (μ) and have a surrounding membrane.

The fusion proteins are separated by their densities, which tend to be greater than that of any other protein present in a transfected cell. That separation by density is typically carried out by use of a centrifuge as is commonly found in biochemistry laboratories through out the world. An illustrative commercially available centrifuge is a Beckman Coulter Avanti™ model J-25 that is used hereinafter for one cushion runs and direct centrifugation. The Beckman Coulter Optima™ XL-100K ultracentrifuge (rotor SW41Ti) was used for the gradient studies. The centrifugation is frequently carried out in the presence of an added differential density-providing solute such as a salt like cesium chloride or a sugar such as sucrose. Combining the homogenate and differential density-providing solute forms a homogenate-solute admixture.

In a particular embodiment, the recombinant fusion proteins comprise, or are preferably made of protein body-inducing sequences (PBIS) linked by a peptide bond to products (e.g., peptides or proteins) of interest (targets). PBIS are protein or amino acid sequences that mediate protein entry and/or accumulation in RPBLAs. Illustrative, non-limiting examples of PBIS include storage proteins or modified storage proteins, as for instance, prolamins or modified prolamins, prolamin domains or modified prolamin domains. Prolamins are reviewed in Shewry et al., 2002 J. Exp. Bot. 53(370):947-958. gamma-Zein, a maize storage protein whose DNA and amino acid residue sequences are shown hereinafter, is one of the four maize prolamins and represents 10-15 percent of the total protein in the maize endosperm. As other cereal prolamins, alpha- and gamma-zeins are biosynthesized in membrane-bound polysomes at the cytoplasmic side of the rough ER, assembled within the lumen and then sequestered into ER-derived PB (Herman et al., 1999 Plant Cell 11:601-613; Ludevid et al., 1984 Plant Mol. Biol. 3:277-234; Torrent et al., 1986 Plant Mol. Biol. 7:93-403).

gamma-Zein is composed of four characteristic domains i) a peptide signal of 19 amino acids, ii) the repeat domain containing eight units of the hexapeptide PPPVHL (SEQ ID NO: 1) (53 aa), iii) the ProX domain where proline residues alternate with other amino acids (29 aa) and iv) the hydrophobic cysteine rich C-terminal domain (111 aa).

The ability of gamma-zein to assemble in ER-derived protein bodies (PBs) is not restricted to seeds. In fact, when gamma-zein-gene was constitutively expressed in transgenic Arabidopsis plants, the storage protein accumulated within ER-derived recombinant PBs in leaf mesophyl cells (Geli et al., 1994 Plant Cell 6:1911-1922). Looking for a signal responsible for the gamma-zein deposition into the ER-derived PB (prolamins do not have KDEL signal), it has been demonstrated that the proline-rich N-terminal domain including the tandem repeat domain was necessary for ER retention and that the C-terminal domain was involved in PB formation. However, the mechanisms by which these domains promote the PB assembly are still unknown. Inasmuch as protein bodies are appropriately so-named only in seeds, similar structures produced in other plant organs and in non-higher plants are referred to generally as recombinant protein body-like assemblies (RPBLAs).

Illustrative other useful prolamin-type sequences are shown in the Table below along with their GenBank identifiers.

| PROTEIN NAME | GENBANK ID |
|---|---|
| α-Zein (22 kD) | M86591 |
| Albumin (32 kD) | X70153 |
| β-Zein (14 kD) | M13507 |
| γ-Zein (27 kD) | X53514 |
| γ-Zein (50 kD) | AF371263 |
| δ-Zein (18 kD) | AF371265 |
| δ-Zein (10 kD) | U25674 |
| 7S Globulin or Vicilin type | NM113163 |
| 11S Globulin or Legumin type | DQ256294 |
| Prolamin 13 kD | AB016504 |
| Prolamin 16 kD | AY427574 |
| Prolamin 10 kD | AF294580 |

Further useful sequences are obtained by carrying out a BLAST search in the all non-redundant GenBank CDS translations+PDB+SwissProt+PIR+PRF (excluding environmental samples) data base as described in Altschul et al., 1997 Nucleic Acids Res. 25:3389-3402 using a query such as those shown below:

| | |
|---|---|
| RX3 | |
| ppppvhlpppvhlpppvhlpppvhlpppvhlpppvhl | SEQ ID NO: 2 |
| pppvhvpppvhlpppp | |
| Alpha- zein | |
| qqqqqflpalsqldvvnpvaylqqqllasnplalanv | SEQ ID NO: 3 |
| aayqqqqqlqqflpalsqlamvnpaayl | |
| Rice prolamin | |
| qqvlspynefvrqqygiaaspflqsatfqlrnnqvwq | SEQ ID NO: 4 |
| qlalvaqqshcqdinivqaiaqqlqlqqfgdly | |

An illustrative modified prolamin includes (a) a signal peptide sequence, (b) a sequence of one or more copies of the repeat domain hexapeptide PPPVHL (SEQ ID NO: 1) of the protein gamma-zein, the entire domain containing eight hexapeptide units; and (c) a sequence of all or part of the ProX domain of gamma-zein. Illustrative specific modified prolamins include the polypeptides identified below as R3, RX3 and P4 whose DNA and amino acid residue sequences are also shown below.

Particularly preferred prolamins include gamma-zein and its component portions as disclosed in published application WO2004003207, the rice rP13 protein and the 22 kDa N-terminal fragment of the maize alpha-zein. The DNA and amino acid residue sequences of the gamma-zein, rice and alpha-zein proteins are shown below.

Gamma-zein of 27 kD

DNA Sequence:

SEQ ID NO: 5

| | |
|---|---|
| atgagggtgt tgctcgttgc cctcgctctc ctggctctcg | 40 |
| ctgcgagcgc cacctccacg catacaagcg gcggctgcgg | 80 |
| ctgccagcca ccgccgccgg ttcatctacc gccgccggtg | 120 |
| catctgccac ctccggttca cctgccacct ccggtgcatc | 160 |
| tcccaccgcc ggtccacctg ccgccgccgg tccacctgcc | 200 |
| accgccggtc catgtgccgc cgccggttca tctgccgccg | 240 |
| ccaccatgcc actaccctac tcaaccgccc cggcctcagc | 280 |
| ctcatcccca gccacaccca tgcccgtgcc aacagccgca | 320 |
| tccaagcccg tgccagctgc agggaacctg cggcgttggc | 360 |
| agcaccccga tcctgggcca gtgcgtcgag tttctgaggc | 400 |
| atcagtgcag cccgacggcg acgccctact gctcgcctca | 440 |
| gtgccagtcg ttgcggcagc agtgttgcca gcagctcagg | 480 |
| caggtggagc cgcagcaccg gtaccaggcg atcttcggct | 520 |
| tggtcctcca gtccatcctg cagcagcagc cgcaaagcgg | 560 |
| ccaggtcgcg gggctgttgg cggcgcagat agcgcagcaa | 600 |
| ctgacggcga tgtgcggcct gcagcagccg actccatgcc | 640 |
| cctacgctgc tgccggcggt gtcccccacg cc | 672 |

Protein Sequence:

```
SEQ ID NO: 6
Met Arg Val Leu Leu Val Ala Leu Ala Leu Leu Ala Leu Ala Ala Ser
    1  5  10  15
Ala Thr Ser Thr His Thr Ser Gly Gly Cys Gly Cys Gln Pro Pro Pro
                          20  25  30
Pro Val His Leu Pro Pro Pro Val His Leu Pro Pro Pro Val His Leu
                  35  40  45
Pro Pro Pro Val His Leu Pro Pro Pro Val His Leu Pro Pro Pro Val
          50  55  60
His Leu Pro Pro Pro Val His Val Pro Pro Pro Val His Leu Pro Pro
  65  70  75  80
Pro Pro Cys His Tyr Pro Thr Gln Pro Pro Arg Pro Gln Pro His Pro
                                  85  90 95
Gln Pro His Pro Cys Pro Cys Gln Gln Pro His Pro Ser Pro Cys Gln
                        100  105  110
Leu Gln Gly Thr Cys Gly Val Gly Ser Thr Pro Ile Leu Gly Gln Cys
                115  120  125
Val Glu Phe Leu Arg His Gln Cys Ser Pro Thr Ala Thr Pro Tyr Cys
        130  135  140
Ser Pro Gln Cys Gln Ser Leu Arg Gln Gln Cys Cys Gln Gln Leu Arg
145  150  155  160
Gln Val Glu Pro Gln His Arg Tyr Gln Ala Ile Phe Gly Leu Val Leu
                                165  170  175
Gln Ser Ile Leu Gln Gln Gln Pro Gln Ser Gly Gln Val Ala Gly Leu
                        180  185  190
Leu Ala Ala Gln Ile Ala Gln Gln Leu Thr Ala Met Cys Gly Leu Gln
                195  200  205
Gln Pro Thr Pro Cys Pro Tyr Ala Ala Ala Gly Gly Val Pro His Ala
        210  215  220
```

RX3

DNA Sequence:

SEQ ID NO: 7

| | |
|---|---|
| atgagggtgt tgctcgttgc cctcgctctc ctggctctcg | 40 |
| ctgcgagcgc cacctccacg catacaagcg gcggctgcgg | 80 |
| ctgccagcca ccgccgccgg ttcatctacc gccgccggtg | 120 |
| catctgccac ctccggttca cctgccacct ccggtgcatc | 160 |
| tcccaccgcc ggtccacctg ccgccgccgg tccacctgcc | 200 |
| accgccggtc catgtgccgc cgccggttca tctgccgccg | 240 |
| ccaccatgcc actaccctac tcaaccgccc cggcctcagc | 280 |
| ctcatcccca gccacaccca tgcccgtgcc aacagccgca | 320 |
| tccaagcccg tgccagacc | 339 |

Protein Sequence:

```
SEQ ID NO: 8
Met Arg Val Leu Leu Val Ala Leu Ala Leu Leu Ala Leu Ala Ala Ser
    1  5  10  15
Ala Thr Ser Thr His Thr Ser Gly Gly Cys Gly Cys Gln Pro Pro Pro
                          20  25  30
Pro Val His Leu Pro Pro Pro Val His Leu Pro Pro Pro Val His Leu
                  35  40  45
Pro Pro Pro Val His Leu Pro Pro Pro Val His Leu Pro Pro Pro Val
          50  55  60
His Leu Pro Pro Pro Val His Val Pro Pro Pro Val His Leu Pro Pro
65  70  75  80
Pro Pro Cys His Tyr Pro Thr Gln Pro Pro Arg Pro Gln Pro His Pro
                                85  90  95
Gln Pro His Pro Cys Pro Cys Gln Gln Pro His Pro Ser Pro Cys Gln
                        100  105  110
Tyr
```

R3

DNA Sequence:

SEQ ID NO: 9

| | |
|---|---|
| atgagggtgt tgctcgttgc cctcgctctc ctggctctcg | 40 |
| ctgcgagcgc cacctccacg catacaagcg gcggctgcgg | 80 |
| ctgccagcca ccgccgccgg ttcatctacc gccgccggtg | 120 |
| catctgccac ctccggttca cctgccacct ccggtgcatc | 160 |
| tcccaccgcc ggtccacctg ccgccgccgg tccacctgcc | 200 |
| accgccggtc catgtgccgc cgccggttca tctgccgccg | 240 |

Protein Sequence:

```
SEQ ID NO: 10
Met Arg Val Leu Leu Val Ala Leu Ala Leu Leu Ala Leu Ala Ala Ser
    1  5  10  15
Ala Thr Ser Thr His Thr Ser Gly Gly Cys Gly Cys Gln Pro Pro Pro
                          20  25  30
Pro Val His Leu Pro Pro Pro Val His Leu Pro Pro Pro Val His Leu
                  35  40  45
Pro Pro Pro Val His Leu Pro Pro Pro Val His Leu Pro Pro Pro Val
          50  55  60
His Leu Pro Pro Pro Val His Val Pro Pro Pro Val His Leu Pro Pro
  65  70  75  80
Pro Pro Cys His Tyr Pro Thr Gln Pro Pro Arg Tyr
                                  85  90
```

P4

DNA Sequence:

SEQ ID NO: 11

| | |
|---|---|
| atgagggtgt tgctcgttgc cctcgctctc ctggctctcg | 40 |
| ctgcgagcgc cacctccacg catacaagcg gcggctgcgg | 80 |
| ctgccagcca ccgccgccgg ttcatctgcc gccgccacca | 120 |
| tgccactacc ctacacaacc gccccggcct cagcctcatc | 160 |
| cccagccaca cccatgcccg tgccaacagc cgcatccaag | 200 |
| cccgtgccag acc | 213 |

Protein Sequence:

```
SEQ ID NO: 12
Met Arg Val Leu Leu Val Ala Leu Ala Leu Leu Ala Leu Ala Ala Ser
1  5  10  15
Ala Thr Ser Thr His Thr Ser Gly Gly Cys Gly Cys Gln Pro Pro Pro
                        20  25  30
Pro Val His Leu Pro Pro Pro Pro Cys His Tyr Pro Thr Gln Pro Pro
                35  40  45
Arg Pro Gln Pro His Pro Gln Pro His Pro Cys Pro Cys Gln Gln Pro
        50  55  60
His Pro Ser Pro Cys Gln Tyr
65  70
```

X10

DNA Sequence:

SEQ ID NO: 13

| | |
|---|---|
| atgagggtgt tgctcgttgc cctcgctctc ctggctctcg | 40 |
| ctgcgagcgc cacctccacg catacaagcg gcggctgcgg | 80 |
| ctgccaatgc cactacccta ctcaaccgcc ccggcctcag | 120 |
| cctcatcccc agccacaccc atgcccgtgc caacagccgc | 160 |
| atccaagccc gtgccagacc | 180 |

Protein Sequence:

```
SEQ ID NO: 14
Met Arg Val Leu Leu Val Ala Leu Ala Leu Leu Ala Leu Ala Ala Ser
    1  5  10  15
Ala Thr Ser Thr His Thr Ser Gly Gly Cys Gly Cys Gln Cys His Tyr
                        20  25  30
Pro Thr Gln Pro Pro Arg Pro Gln Pro His Pro Gln Pro His Pro Cys
                  35 40  45
Pro Cys Gln Gln Pro His Pro Ser Pro Cys Gln Tyr
          50  55  60
```

rP13—rice prolamin of 13 kD homologous to the clone—(GenBank AB016504) Sha et al., 1996 Biosci. Biotechnol. Biochem. 60(2):335-337; Wen et al., 1993 Plant Physiol. 101(3):1115-1116; Kawagoe et al., 2005 Plant Cell 17(4):1141-1153; Mullins et al., 2004 J. Agric. Food Chem. 52(8):2242-2246; Mitsukawa et al., 1999 Biosci. Biotechnol. Biochem. 63(11):1851-1858

Protein Sequence:

SEQ ID NO: 15

mkiifvfallaiaacsasaqfdvlgqsyrqyqlqspvllqqqvlspynef
vrqqygiaaspflqsatfqlrnnqvwqqlalvaqqshcqdinivqaiaqq
lqlqqfgdlyfdrnlaqaqallafnvpsrygiypryygapstittlggvl

DNA Sequence:

SEQ ID NO: 16

atgaagatcattttcgtctttgctctccttgctattgctgcatgcagcgc
ctctgcgcagtttgatgttttaggtcaaagttataggcaatatcagctgc
agtcgcctgtcctgctacagcaacaggtgcttagcccatataatgagttc
gtaaggcagcagtatggcatagcggcaagccccttcttgcaatcagctac
gtttcaactgagaaacaaccaagtctggcaacagctcgcgctggtggcgc
aacaatctcactgtcaggacattaacattgttcaggccatagcgcagcag
ctacaactccagcagtttggtgatctctactttgatcggaatctggctca
agctcaagctctgttggcttttaacgtgccatctagatatggtatctacc
ctaggtactatggtgcacccagtaccattaccacccttggcggtgtcttg

22aZt N-terminal fragment of the maize alpha-zein of 22 kD—(GenBank V01475) Kim et al., 2002 Plant Cell 14(3):655-672; Woo et al., 2001 Plant Cell 13(10):2297-2317; Matsushima et al., 1997 Biochim. Biophys. Acta 1339(1):14-22; Thompson et al., 1992 Plant Mol. Biol. 18(4):827-833.

Protein Sequence (Full Length):

SEQ ID NO: 17

matkilallallalfvsatnafiipqcslapsaiipqflppvtsmgfehl
avqayrlqqalaasvlqqpinqlqqqslahltiqtiatqqqqqflpalsq
ldvvnpvaylqqqllasnplalanvaayqqqqqlqqflpalsql

DNA Sequence (Full Length):

SEQ ID NO: 18

atggctaccaagatattagccctccttgcgcttcttgccctttttgtgag
cgcaacaaatgcgttcattattccacaatgctcacttgctcctagtgcca
ttataccacagttcctcccaccagttacttcaatgggcttcgaacaccta
gctgtgcaagcctacaggctacaacaagcgcttgcggcaagcgtcttaca
acaaccaattaaccaattgcaacaacaatccttggcacatctaaccatac
aaaccatcgcaacgcaacagcaacaacagttcctaccagcactgagccaa
ctagatgtggtgaaccctgtcgcctacttgcaacagcagctgcttgcatc
caacccacttgctctggcaaacgtagctgcataccaacaacaacaacaat
tgcagcagtttctgccagcgctcagtcaacta

Examples of proteins of interest include any protein having therapeutic, nutraceutical, biocontrol, or industrial uses, such as, for example monoclonal antibodies (mAbs such as IgG, IgM, IgA, etc.) and fragments thereof, antigens for vaccines (human immunodeficiency virus, HIV; hepatitis B pre-surface, surface and core antigens, gastroenteritis corona virus, etc.), hormones (calcitonin, growth hormone, etc.), protease inhibitors, antibiotics, collagen, human lactoferrin, cytokines, industrial enzymes (hydrolases, glycosidases, oxido-reductases, and the like). Illustrative DNA and amino acid residue sequences for illustrative proteins of interest are provided below.

Salmon Calcitonin Genbank BAC57417

Protein Sequence:

| | |
|---|---|
| kcsnlstcvlgklsqelhklqtyprtntgsgtpg | SEQ ID NO: 19 |

DNA Sequence:

SEQ ID NO: 20

aagtgctccaacctctctacctgcgttcttggtaagctctctcaggagct
tcacaagctccagacttaccctagaaccaacactggttccggtacccct
ggt

hEGF—Construction based on the GenBank AAF85790 without the signal peptide

Protein Sequence:

SEQ ID NO: 21

nsdsecplshdgyclhdgvcmyiealdkyacncvvgyigercqyrdlkww
elr

DNA Sequence:

SEQ ID NO: 22

aactctgattcagaatgcccactcagtcacgacggatattgtcttcacga
tggggtatgcatgtacatcgaggccttggacaagtacgcatgtaattgtg
tagtgggatacattggtgaacgctgtcagtatcgagacttgaaatggtgg
gagcttaggtga

hGH—Construction based in the P01241 without the signal peptide

Protein Sequence:

SEQ ID NO: 23

fptiplsrlfdnamlrahrlhqlafdtyqefeeayipkeqkysflqnpqt
slcfsesiptpsnreetqqksnlellrisllliqswlepvqflrsvfans
lvygasdsnvydllkdleegiqtlmgrledgsprtgqifkqtyskfdtns
hnddallknygllycfrkdmdkvetflrivqcrsvegscgf

DNA Sequence:

Using Plant-Preferred Codons

SEQ ID NO: 24

tttcctactattcctttatctcgactcttcgacaacgctatgcttagagc
gcaccgcctacaccagcttgcattcgatacataccaagagtttgaagagg
cctacattcctaaggaacagaagtattcatttctacagaatcctcaaaca
agtctttgtttctctgagtccatccctactccctcgaacagggaggaaac
tcaacagaagagtaatttggagttgcttcgcatatccttgttactcatac
aatcttggcttgaacccgttcaattcttaaggtcagtgtttgccaattca
cttgtatatggtgcatcagattcgaatgtatatgacctattgaaagactt
ggaagagggtattcaaacacttatgggacgtttggaagatgggtctccaa
ggacgggacaaatcttcaaacagacttacagcaaattcgatacaaattca
cataacgacgatgcattacttaagaactatgggttgctttattgtttccg
gaaggatatggacaaagtcgagacctttctgagaattgttcaatgtagat
ctgtagaaggttcctgtggattctga

Using Native Codons

SEQ ID NO: 25

ttcccaaccattcccttatccaggctttttgacaacgctatgctccgcgc
ccatcgtctgcaccagctggcctttgacacctaccaggagtttgaagaag
cctatatcccaaaggaacagaagtattcattcctgcagaacccccagacc
tccctctgtttctcagagtctattccgacaccctccaacagggaggaaac
acaacagaaatccaacctagagctgctccgcatctccctgctgctcatcc
agtcgtggctggagcccgtgcagttcctcaggagtgtcttcgccaacagc
ctggtgtacggcgcctctgacagcaacgtctatgacctcctaaaggacct
agaggaaggcatccaaacgctgatggggaggctggaagatggcagccccc
ggactgggcagatcttcaagcagacctacagcaagttcgacacaaactca
cacaacgatgacgcactactcaagaactacgggctgctctactgcttcag
gaaggacatggacaaggtcgagacattcctgcgcatcgtgcagtgccgct
ctgtggagggcagctgtggcttctga

In another embodiment, the recombinant fusion protein further comprises in addition to the sequences of the PBIS and product of interest, a spacer amino acid sequence. The spacer amino acid sequence can be an amino acid sequence cleavable by enzymatic or chemical means or not cleavable. In a particular embodiment, the spacer amino acid sequence is placed between said PBIS and product of interest. An illustrative an amino acid sequence is cleavable by a protease such as an enterokinase, Arg—C endoprotease, Glu—C endoprotease, Lys—C endoprotease, Factor Xa and the like. Alternatively, an amino acid sequence is encoded that is specifically cleavable by a chemical reagent, such as, for example, cyanogen bromide that cleaves at methionine residues.

In further embodiment, the nucleic acid sequence used for transformation purposes is as disclosed according to co-assigned patent application WO 2004003207 that includes a cleavable amino acid residue sequence between the PBIS and the polypeptide of interest. Further, in yet another embodiment, the nucleic acid sequence is as disclosed according to patent application WO 2004003207, but the nucleic acid sequence coding for the cleavable amino acid sequence is absent.

In a preferred embodiment, the fusion proteins are prepared according to a method that comprises transforming the host cell system such as an animal, animal cell culture, plant, plant cell culture, fungi or algae with a nucleic acid sequence comprising (i) a first nucleic acid coding for a PBIS that is operatively linked in frame to (ii) a second nucleic acid sequence comprising the nucleotide sequence coding for a product of interest; that is, the nucleic acid sequence that encodes the PBIS is chemically bonded to the sequence that encodes the polypeptide of interest such that both polypeptides are expressed from their proper reading frames. Upon expression, the resulting fusion protein accumulates in the transformed host-system as high density recombinant protein body-like assemblies. In one embodiment, the 3′ end of the first nucleic acid sequence (i) is linked (bonded) to the 5′ end of the second nucleic acid sequence (ii). In another embodiment, the 5′ end of the first nucleic acid sequence (i) is linked (bonded) to the 3′ end of the second nucleic acid sequence (ii). In another embodiment, the PBIS comprises a storage protein or a modified storage protein, a fragment or a modified fragment thereof.

In another particular embodiment, a fusion protein is prepared according to a method that comprises transforming the host cell system such as an animal, animal cell culture, plant, plant cell culture, fungi or algae with a nucleic acid sequence comprising, in addition to the nucleic acid sequences (i) and (ii) previously mentioned, an in frame nucleic acid sequence (iii) that codes for a spacer amino acid sequence. The spacer amino acid sequence can be an amino acid sequence cleavable by enzymatic or chemical means or not cleavable, as noted before. In one particular embodiment, the nucleic acid sequence (iii) is placed between said nucleic acid sequences (i) and (ii), e.g., the 3′ end of the third nucleic acid sequence (iii) is linked to the 5′ end of the second nucleic acid sequence (ii). In another embodiment, the 5′ end of the third nucleic acid sequence (iii) is linked to the 3′ end of the second nucleic acid sequence (ii).

As used herein, the term plant host cell comprises plants, including both monocots and dicots, and, specifically, cereals (e.g., maize, rice, oats, and the like), legumes (e.g., soy, and the like), a cruciferous plant (e.g., Arabidopsis thaliana, colza, and the like) and a solanaceous plant (e.g., potato, tomato, tobacco, and the like).

A plant host system also encompasses plant cells. Plant cells include suspension cultures, embryos, meristematic regions, callus tissue, leaves, roots, shoots, gametophytes, sporophytes, pollen, seeds and microspores. A plant host cell system can be at various stages of maturity and can be grown in liquid or solid culture, or in soil or suitable medium in pots, greenhouses or fields. Expression in plant host cell systems can be transient or permanent. Plant host cell system also refers to any clone of such plant, seed, selfed or hybrid progeny, propagule whether generated sexually or asexually, and descendants of any of these, such as cuttings or seeds.

Transformation of plant cells using Agrobacterium tumefaciens is typically best carried out on dicotyledonous plants. Monocots are usually most readily transformed by so-called direct gene transfer of protoplasts. Direct gene transfer is usually carried out by electroportation, by polyethyleneglycol-mediated transfer or bombardment of cells by microprojectiles carrying the needed DNA. These methods of transfection are well-known in the art and need not be further discussed herein. It is also noted that at lest rice and maize can be transformed by Agrobacterium. Methods of regenerating whole plants from transfected cells and protoplasts are also well-known, as are techniques for obtaining a desired protein from plant tissues. See, also, U.S. Pat. No. 5,618,988 and No. 5,679,880 and the citations therein.

A contemplated method can also include the recovery and solubilization of a recombinant fusion protein. Thus, for example, RPBLAs collected from a step-density gradient or one-step density cushion are suspended in a buffered solution containing a reducing agent and centrifuged. The pellet is discarded and the recombinant protein recovered from the supernatant to be further purified as desired such as by classical chromatographic methods.

Without further elaboration, it is believed that one skilled in the art can, using the preceding description and the detailed examples below, utilize the present invention to its fullest extent. The following preferred specific embodiments are, therefore, to be construed as merely illustrative, and not limiting of the remainder of the disclosure in any way whatsoever.

Experimental Procedure

### EXAMPLE 1

### Plasmid Construction for Plant Transformation

The coding sequences of T20 and human epidermal growth factor (hEGF) were obtained synthetically and were modified in order to optimize its codon usage for expression in plants.

The first strand of the cDNA sequence encoding the 36 amino acids of T20 was obtained by chemical oligonucleotide synthesis, and the sequence corresponding to the Factor Xa specific cleavage site and enzyme restriction site were added at 5′ end of the sequence. This synthetic construction was purified by polyacrylamide denaturing gel.

SEQ ID NO: 26

5′ CATGGGCATTGAAGGTAGATATACTTCCCTTATTCATTCACTGATCG
AAGAGTCTCAGAACCAACAAGAGAAGAATGAGCAAGAACTCCTTGAGCTG
GACAAGTGGGCTTCTTTGTGGAACTGGTTCTGATAAG 3′

The double-stranded cDNA was obtained by PCR using specific T20 primers containing restriction sites for further cloning.

Primers:

| | |
|---|---|
| V20Forward | |
| 5′CATGCCA TGGGC ATTGAAGGTAG-3′ | SEQ ID NO: 27 |
| V20Reverse | |
| 5′CGCGGATCCTTATCAGAACC AGTTCCACA-3′ | SEQ ID NO: 28 |

The synthetic gene encoding the 53 amino acids of active hEGF was obtained by primer overlap extension PCR method, using 4 oligonucleotides of around 60 bases, with 20 overlapping bases. The synthetic hEGF cDNA included a 5′ linker sequence corresponding to the Factor Xa specific cleavage site. The oligonucleotides were purified by polyacrylamide denaturing gel.

EGF1:

SEQ ID NO: 29

5′CATGCCATGGGAATTGAGGGTAGGAACTCTGATTCAGAATGCCCACTC
AGTCACGACGGA TATT 3′

EGF2:

SEQ ID NO: 30

5′ACTTGTCCAAGGCCTCGATGTACATGCATACCCCATCGTGAAGACAA
TATCCGTCGTGACTGAGT 3′

EGF3:

SEQ ID NO: 31

5′CATCGAGGCCTTGGACAAGTACGCATGTAATTGTGTAGTGGGATAC
ATTGGTGAACGCTGTCAGT 3′

EGF4:

SEQ ID NO: 32

5′TCAGGATCCTTATCACCTAAGCTCCCACCATTTCAAGTCTCGATACT
GACAGCGTTCACCAATGT 3′

The synthetic gene encoding the 191 amino acids of active hGH was obtained by primer overlap extension PCR met Example 2: Plasmid construction hod, using 15 oligonucleotides of about 60 bases, with 20 overlapping bases. The synthetic hGH cDNA included a 5′ linker sequence corresponding to the enterokinase specific cleavage site. The oligonucleotides were purified by polyacrylamide denaturing gel.

hGH1:

SEQ ID NO:33

5′GTCCATGGACGATGATGACAAGTTTCCTACTATTCCTTTATCTCGACT
CTTCGACAACGCTA3′

hGH2:

SEQ ID NO:34

5′GTATCGAATGCAAGCTGGTGTAGGCGGTGCGCTCTAAGCATAGCGTTG
TCGAAGAGTCGA3′

hGH3:

SEQ ID NO:35

5′CACCAGCTTGCATTCGATACATACCAAGAGTTTGAAGAGGCCTACATT
CCTAAGGAACAGAA3′

hGH4:

SEQ ID NO:36

5′GAGAAACAAAGACTTGTTTGAGGATTCTGTAGAAATGAATACTTCTGT
TCCTTAGGAATGTA3′

hGH5:

SEQ ID NO:37

5′CAAACAAGTCTTTGTTTCTCTGAGTCCATCCCTACTCCCTCGAACAGG
GAGGAAACTCAACA3′

hGH6:

SEQ ID NO:38

5′GAGTAACAAGGATATGCGAAGCAACTCCAAATTACTCTTCTGTTGAGT
TTCCTCCCTGTT3′

hGH7:

SEQ ID NO:39

5′CTTCGCATATCCTTGTTACTCATACAATCTTGGCTTGAACCCGTTCAA
TTCTTAAGGT3′

hGH8:

SEQ ID NO:40

5′CGAATCTGATGCACCATATACAAGTGAATTGGCAAACACTGACCTTAA
GAATTGAACGGGT3′

hGH9:

SEQ ID NO: 41

5′GTATATGGTGCATCAGATTCGAATGTATATGACCTATTGAAAGACTTG
GAAGAGGGTATTCA3′

hGH10:

SEQ ID NO: 42

5′CCGTCCTTGGAGACCCATCTTCCAAACGTCCCATAAGTGTTTGAATAC
CCTCTTCCAAGTCT3′

hGH11:

SEQ ID NO: 43

5′GATGGGTCTCCAAGGACGGGACAAATCTTCAAACAGACTTACAGCAAA
TTCGATACAAATT3′

hGH12:

SEQ ID NO: 44

5′GCAACCCATAGTTCTTAAGTAATGCATCGTCGTTATGTGAATTTGTAT
CGAATTTGCTGT3′

hGH13:

SEQ ID NO: 45

5′CTTAAGAACTATGGGTTGCTTTATTGTTTCCGGAAGGATATGGACAAA
GTCGAGACCTTTCT3′

hGH14:

SEQ ID NO: 46

5′GAATCCACAGGAACCTTCTACAGATCTACATTGAACAATTCTCAGAAA
GGTCTCGACTTTGT3′

hGH15:

SEQ ID NO:47

5′TCAGGATCCTTATTATCAGAATCCACAGGAACCTTCTA3′

Synthetic T20 and hEGF cDNA were purified from agarose gel (Amersham) and cloned into pGEM vector (Promega). The RX3 cDNA fragment (coding for an N-terminal domain of gamma-zein) containing cohesive ends of BspHI and NcoI, was inserted into the vector pCKGFPS65C (Reichel et al., 1996 Proc. Natl. Acad. Sci. USA 93:5888-5893) previously digested with NcoI (as described in patent application WO2004003207). The sequences coding for T20 and EGF were fused in frame to the RX3 sequence. The constructs RX3-T20 and RX3-EGF were prepared by substitution of the GFP coding sequence for the T20 and EGF synthetic gene.

The resulting constructs named pCRX3T20 and pCRX3EGF contained a nucleic acid sequence that directs transcription of a protein as the enhanced ³⁵S promoter, a translation enhancer as the tobacco etch virus (TEV), the T20 and EGF coding sequences and the 3′ polyadenylation sequences from the cauliflower mosaic virus (CaMV). Effective plant transformation vectors p19RX3T20 and p19RX3EGF were ultimately obtained by inserting the HindIII/HindIII expression cassettes into the binary vector pBin19 (Bevan, 1984 Nucleic Acids Research 12:8711-8721).

The cDNA encoding the hGH was fused to the RX3 N-terminal gamma-zein coding sequence (patent WO2004003207) and inserted into a pUC18 derived plasmid containing the enhanced CaMV ³⁵S promoter and 3′ ocs terminator. The expression cassette from the pUC18 derived plasmid named pUC18RX3hGH, that contained the corresponding fusion protein RX3-hGH sequence, was introduced in the pBin19 binary vector (Bevan, 1984 Nucleic Acids Research 12:8711-8721).

The cDNA encoding the alpha zein of 22 kD (22aZ) and the rice prolamin of 13 kD (rP13) were amplified by RT-PCR from a cDNA library from maize W64A and Senia rice cultivar, respectively. The oligonucleotides used in the PCR reaction were:

| | |
|---|---|
| 22aZ-5′ | |
| 5′GAGGATCCGCATGGCTACCAAGATATTAGCCCT3′ | SEQ ID NO: 48 |
| 22aZ-3′ | |
| 5′CATTCATGATTCCGCCACCTCCACCAAAGATGGCA | SEQ ID NO: 49 |
| CCTCCAACGATGG3′ | |
| Rice13Prol-5′ | |
| 5′GAGTCGACGGATCCATGAAGATCATTTTCGTCTTT | SEQ ID NO: 50 |
| GCTCTCC3′ | |
| Rice13Prol-3′: | |
| 5′CATCCATGGTTCCGCCACCTCCACCCAAGACACCG | SEQ ID NO: 51 |
| CCAAGGGTGGTAATGG3′ | |

The corresponding PCR fragments were cloned in the pCRII vector (Invitrogen), sequenced and cloned in pUC18 vectors containing the enhanced CaMV ³⁵S promoter, the TEV sequence and 3′ ocs terminator. The pCRII-rP13 was digested by SalI and NcoI, and cloned in the pUC18RX3Ct, pUC18RX3hGH and pUC18RX3EGF plasmids digested by the same enzymes to obtain respectively: pUC18rP13Ct, pUC18rP13hGH and pUC18rP13EGF. The pCRII-22aZ was digested by SalI/NcoI and cloned in the pUC18RX3Ct and pUC18RX3EGF plasmid digested by the same enzymes to obtain pUC1822aZtCt and pUC1822aZtEGF respectively. The pCRII-22aZ was also digested by SalI/RcaI and cloned in the pUC18RX3hGH plasmid digested by SalI/NcoI to obtain the clone pUC1822aZhGH. Finally, all these pUC18-derived vectors were cloned in pCambia 5300 by HindIII/EcoRI.

### EXAMPLE 2

### Plasmid Construction for Animal and Yeast Cell Transformation

Animal Cells

The synthetic gene corresponding to the mature calcitonin sequence (Ct, WO2004003207) and EGF sequences as well the cDNA encoding the hGH were fused to the RX3 N-terminal gamma-zein coding sequence (patent WO2004003207) and were introduced into the vector pUC18. SalI-BamHI restriction fragments from the pUC18 derived plasmids pUC18RX3Ct, pUC18RX3EGF and pUC18RX3hGH, containing the corresponding fusion protein RX3-Ct, RX3-EGF and RX3-hGH sequences, were introduced in the vector pcDNA3.1—(Invitrogen) restricted with Xho I-Bam HI. In the resulting constructs named p3.1RX3CT, p3.1RX3EGF and p3.1RX3hGH, the fusion protein sequences were under the CMV promoter and the terminator pA BGH.

Yeast Cells

SalI (blunt ended)—BamHI restriction fragments from the pUC18 derived plasmids described above, containing the corresponding fusion protein RX3-EGF and RX3-hGH sequences were introduced in the vector pYX243 (R&D Systems) restricted with EcoRI (blunt ended)—Bam HI. In the resulting constructs named, respectively, c117 and c118, the fusion protein sequences were under the inducible GAL promoter.

### EXAMPLE 3

### Host Transformation

Yeast

The Saccharomyces cerevisiae strain leu2) was transformed with the plasmid constructs c117 and c118 by the LiAc method (Ito et al. 1983, J. Bacteriol. 153:163-168) and transformants were selected on Leu⁻ plates. Expression analyses were made by growing the transformants in a galactose-containing medium.

Plant Material

Tobacco (Nicotiana tabacum var. Wisconsin) plants were grown in an in vitro growth chamber at 24-26° C. with a 16 hour photoperiod. Adult plants were grown in greenhouse between at 18-28° C., humidity maintained between 55 and 65% with average photoperiod of 16 hours.

Plantlets for Agroinfiltration (Vaquero et al., 1999 Proc. Natl. Acad. Sci., USA 96(20):11128-11133; Kapila et al., 1997 Plant Sci. 122:101-108) method were grown from seeds for 4-6 weeks in the in vitro conditions described above.

Tobacco Stable Transformation

The binary vectors were transferred into LBA4404 strain of A. tumefaciens. Tobacco (Nicotiana tobaccum, W38) leaf discs were transformed as described by Draper and Hamil 1988. In: Plant Genetic Transformation and Gene Expression. A Laboratory Manual (Eds. Draper, J., Scott, R., Armitage, P. and Walden, R.), Blackwell Scientific Publications. Regenerated plants were selected on medium containing 200 mg/L kanamycin and transferred to a greenhouse. Transgenic tobacco plants having the highest transgene product levels were cultivated in order to obtain T1 and T2 generations.

Recombinant protein level was detected by immunoblot. Total protein extracts from tobacco leaves were quantified by Bradford assay, separated onto 15% SDS-PAGE and transferred to nitrocellulose membranes using a Mini Trans-Blot Electrophoretic Transfer Cell (Bio Rad). Membranes were incubated with gamma-zein antiserum (dilution 1/7000) (Ludevid et al. 1985, Plant Science 41:41-48) and were then incubated with horseradish peroxidase-conjugated antibodies (dilution 1/10000, Amersham Pharmacia). Immunoreactive bands were detected by enhanced chemiluminescence (ECL western blotting system, Amersham Pharmacia).

Tobacco Agroinfiltration

Plantlets for Agroinfiltration method were grown from seeds for 4-6 weeks in an in vitro growth chamber at 24-26° C. with a 16 hour photoperiod.

A. tumefaciens strain LB4404 containing a desired construct was grown on LB medium (Triptone 10 g/l, yeast extract 5 g/l, NaCl 10 g/l) supplemented with kanamycin (50 mg/l) and rifampicine (100 mg/l) at 28° C. with shaker (250 rpm) overnight (about 18 hours). Agrobacteria were then inoculated in 30 ml of LB also supplemented with kanamycin (50 mg/l) and rifampicine (100 mg/l). After overnight culture at 28° C. (about 18 hours), agrobacterial cells were collected by centrifugation for 10 minutes at 3000×g and resuspended in 10 ml of liquid MS medium with MES (Sigma Chemical) 4.9 g/l and sucrose 30 g/l at pH 5.8. Bacterial culture was adjusted to a final OD₆₀₀ of 0.1 for agroinfiltration. Then, cell culture was supplemented with acetosyringone to a final concentration of 0.2 mM and incubated for 90 minutes at 28° C.

For agroinfiltration, the plantlets were totally covered with the suspension and vacuum was applied (100 KPa) for 5-6 seconds. The suspension was removed and plantlets maintained in a growth chamber at 24-26° C. under a photoperiod of 16 hours for four days. The plantlet material was recovered and total protein extraction analyzed by immunoblot using anti-gamma-zein antibody.

Animal Cell Transformation

The constructs p3.1RX3.Ct, p3.1RX3.EGF and p3.1RX3.hGH were introduced in 293T, Cos1 or CHO cultured mammalian cells by the lipofectamine-based transfection method (Invitrogen). Cells transfected with plasmid pECFP-N1 (Clontech) containing the gene sequence of an enhanced cyan fluorescent modified GFP, were used as a control.

### EXAMPLE 4

### Protein Extraction from Tobacco Leaves

Fresh Plant Material

Plant material (wet or dry tobacco leaves or plantlets) was ground in liquid nitrogen and homogenized with extraction buffer T containing Tris-HCl 50 mM pH 8, 200 mM dithiothreitol (DTT) and protease inhibitors [10 μM Aprotinin, 1 μM pepstatin, 100 μM leupeptine, 100 μM phenylmethylsulphonyl floride (PMSF) and 100 μM E64 (Sigma Chemical)]. The homogenates were centrifuged at 10000×g for 30 minutes at 4° C. to remove insoluble material. Total soluble proteins (TSP) were quantified using Bradford protein assay (BioRad).

Dry Tobacco Leaves and Protein Extraction

Adult transgenic and wild type tobacco leaves were dried in a 37° C. room for two weeks in a filter paper. After two weeks, leaves were cut up and stored at room temperature for 5 months. Total soluble protein of dry material was extracted as described for fresh material and was analyzed by western blot.

### EXAMPLE 5

### RPBLAs Preparation

Homogenization

Fresh and dried transgenic tobacco leaves and agroinfiltrated tobacco plantlets (transient transformation) were ground in a mortar and pestle at 0° C. in a PBP extraction buffer containing Tris 100 mM pH 8, KCl 50 mM, MgCl₂ 6 mM, EDTA 10 mM supplemented with 10% sucrose and protease inhibitors (PMSF, Leupeptine, Aprotinine, E-64). The homogenate was additionally ground using a polytron (IKA T25 Basic, 24.000 rpm) with a small rotor (7.5 mm diameter), about ten times for 3-4 seconds in ice. The solid material was removed by filtering through four layers of Miracloth (22-24 micrometer) (Calbiochem) to remove unruptured tissue and cells.

### EXAMPLE 6

### Proteins from Animal Cells

Transfected cells were recovered from culture plates by scraping and they were suspended in the homogenization B medium (10 mM Tris-HCl pH 8.0, 0.9% NaCl, 5 mM EDTA with protease inhibitors). The suspension was taken into a 5 ml syringe fitted with a 23 gauge needle and it was expelled approximately 30 times. Cell rupture was monitored by a phase contrast microscope. The homogenate was loaded on a step sucrose gradient and centrifuged as described for tobacco leaf homogenates.

The accumulation of fusion proteins in the transiently transfected cells was analyzed by Western blot, using the gamma-zein antibodies raised against the gamma-zein. After 48 hours of transfection, total soluble cell proteins were extracted with buffer A (100 mM Tris-HCl pH 8.0, 150 mM NaCl, 5 mM EDTA, 0.5% SDS, 0.5% Triton X-100, 2% 2-mercaptoethanol and protease inhibitors). Aliquots of the cell incubation media were precipitated and stored at −20° C. Proteins extracted from equivalent amounts of transfected cells and media were separated by SDS polyacrylamide gel electrophoresis and transferred to nitrocellulose sheets for immunodetection.

### EXAMPLE 7

### Separation by Discontinuous Gradient

A homogenate was centrifuged at 50×g for 5 minutes at 4° C. to obtain a clarified extract. For discontinuous sucrose gradient, this clarified homogenate (supernatant) was layered onto a step gradient composed of 2.5 ml of 20%-30%-42%-56% (w/w) or 42%-49%-56%-65% (w/w) sucrose in buffer PBP and it was centrifuged (Beckman Coulter Optima™ XL-100K ultracentrifuge) at 80,000×g in a swinging-bucket rotor (SW41Ti) at 4° C. for 120 minutes without brake.

The supernatant, interphases and pellet fractions were collected. Equivalent aliquots of supernatant, interphase fractions and pellet were precipitated with 15% TCA and analyzed by SDS-PAGE and immunoblot by using specific antibodies against the fusion expressed proteins. Proteins RX3-EGF, RX3-T20, RX3-Ct, and RX3-INF were detected by Western blot using the gamma-zein antibody. The electrophoretic gels were analyzed by silver staining according to Morrissey et al., 1981 Anal. Biochem. 117:307-310 to evaluate the enrichment of recombinant protein vs. contaminant proteins inside PBs.

### EXAMPLE 8

### Separation by One Step Cushion

Homogenate prepared as described above, was centrifuged onto a 42% (w/w) sucrose cushion of 8 ml (1.18 g/cm³) for 120 minutes at 24,000 g at 4° C. Supernatant, interface and pellet fractions were recovered. RPBLAs sediment at the bottom of the cushion. For protein analysis, equivalent aliquots of these fractions were precipitated in 15% TCA and samples were separated on 15% SDS-PAGE and analyzed by silver staining. Recombinant proteins present in PB fraction were detected by immunoblot using gamma-zein antibody.

### EXAMPLE 9

### Recombinant Protein Recovery from Isolated RPBLAs

RPBLAs isolated from the 42%-56% (w/w) interphase of step sucrose gradients or isolated by density cushion of 42% (w/w) of sucrose were washed in PBP Buffer and recovered by a brief centrifugation for 5 minutes at 16000×g. Recombinant proteins accumulated inside RPBLAs were solubilized in one volume of SB buffer containing sodium borate 12.5 mM pH 8, 0.1% SDS and 2% 2-mercaptoethanol. The solution was incubated overnight (about 18 hours) at 37° C. One aliquot was centrifuged 10 minutes at 16000×g at room temperature and the supernatant and pellet were analyzed by SDS-PAGE and Western blot to evaluate complete protein solubilization.

### EXAMPLE 10

### Proteins from Transfected Yeast Cells

S. cerevisiae expressing recombinant fusion proteins were pelleted. Aliquots of the respective incubation media were precipitated and stored at −20° C. to be analyzed. The cell pellets were also frozen and after thawing, the cells were broken by standard methods using glass beads and medium Y (50 mM HCl-Tris pH 8.0, 150 mM NaCl, 5 mM EDTA, 200 mM DTT and protease inhibitors). Equivalent amounts of both, cells and media, were analyzed by SDS-PAGE and immunoblot by using specific antibodies against the recombinant expressed proteins.

The disruption method applied to isolate organelles from transformed yeast cells was based on the gentle lysis of spheroplasts described in Zinser et al., 1995 Yeast, 11:493-536. Thirty mL of cultured transformed yeast cells (DO600 around 0.5) were pelleted, washed with 1 M sorbitol and suspended in 1 mL of spheroplasting buffer (1 M sorbitol, 50 mM potassium phosphate pH 7.5, 14 mM 2-mercaptoethanol) containing 100 units/ml of zymolase. Spheroplast formation was allowed to proceed for 20-30 minutes at 30° C. with occasional gentle agitation. After sedimentation at 1000 g for 6 minutes, the spheroplasts were washed with spheroplasting buffer without 2-mercaptoethanol, and resuspended in 0.5 mL of ice-cold lysis buffer (0.3 M sorbitol, 10 mM triethanolamine, 1 mM EDTA and protease inhibitors). After 20 minutes on ice with occasional gentle agitation, lysates were adjusted to a final concentration of 1.0 M sorbitol. The lysates were loaded on a step sucrose gradient and centrifuged as described for tobacco leaf homogenates. Fractions were analyzed by SDS-PAGE and immunoblot.

Results

### EXAMPLE A

### Isolation (Purification) of RPBLAs by Density Gradient from Transgenic Plant Vegetative Tissues

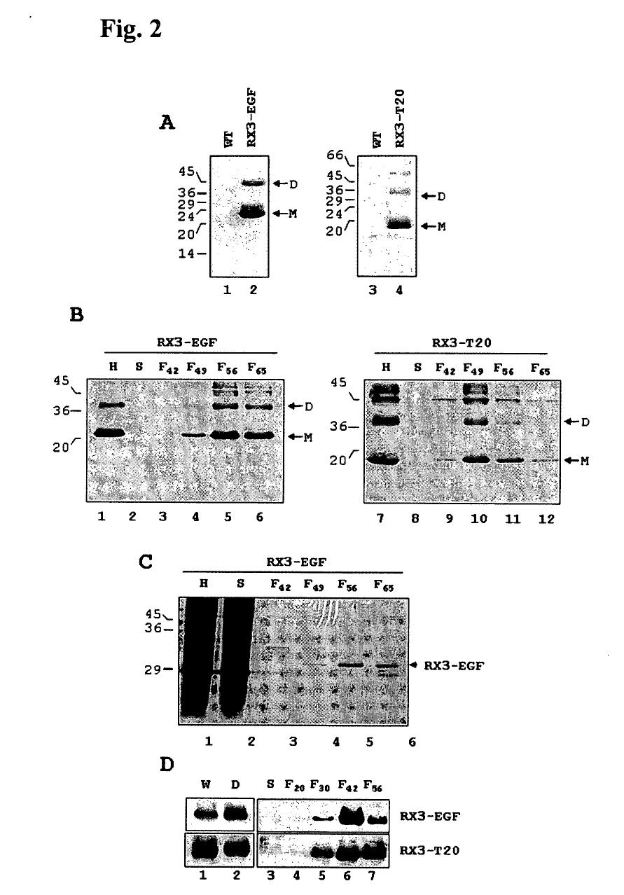

The genes coding for RX3-EGF and RX3-T20 gamma-zein derived fusion proteins were introduced in tobacco plants via Agrobacterium tumefaciens. Transformed plants were analyzed by immunoblot to determine those plants with higher recombinant protein expression. FIG. 2A shows the pattern of both RX3EGF and RX3T20 proteins. It should be noted that both recombinant proteins appear correctly accumulated in all transgenic lines. The predominant lower bands correspond to the monomer forms of fusion proteins and the higher bands to the dimers. The fusion proteins usually accumulate as multimers and the amount of monomers and oligomers detected in the immunoblots depends on the disulfide bond reduction level.

Tobacco leaf extracts were loaded on density step gradients and the accumulation of recombinant proteins in the different fractions was analyzed by immunoblot (FIG. 2B). The results shown in FIG. 2B indicate that RX3-EGF appeared in fractions corresponding to dense RPBLAs. Most of these organelles exhibited densities higher than 1.2632 (F56, lane 6) and a significant portion of them show a density higher than 1.3163 g/cm³ (F65, lane 7). The RX3-T20 fusion protein was present in the interphase 49%-56% sucrose (lane 12), indicating that RPBLAs containing RX3-T20 have densities higher than 1.2241 g/cm³, a significant portion of them being more dense than 1.2632, and a significant proportion had densities greater than 1.2632 (lane 13).

These novel RPBLAs formed in tobacco leaves exhibit densities in the range of the natural maize protein bodies (Ludevid et al., 1984 Plant Mol. Biol. 3:227-234; Lending et al., 1989 Plant Cell 1:1011-1023), or are even more dense. It should be noted that RX3-T20 PBs are rather (slightly) less dense than RX3-EGF PBs, this is attributed to some specific characteristics of the protein of interest. Therefore, although RPBLAs accumulating recombinant fusion proteins have high densities relative to usually present soluble cell portions, the features of the protein fused to RX3 domain can determine variations in such density.

It was estimated that more than 90 percent of both recombinant proteins were recovered in the dense RPBLAs fractions and pellet (see FIG. 2B). Thus, isolation of RPBLAs by density appears to be a useful system to purify (concentrate) the fusion proteins.

To evaluate the purification of the recombinant protein RX3-EGF by RPBLAs isolation, the different density fractions were analyzed by silver stain (FIG. 2C). As can be seen in the stained gel, more than 90 percent of tobacco endogenous proteins were located in the soluble (S) and the interphase fractions (F422 and F49) of the gradient, the fractions in which, RX3-EGF protein was absent or barely detected (see FIG. 2B). Thus, soluble proteins and the bulk of proteins present in less dense organelles could be discarded by selecting one or two fractions (F56 and F65) of the gradient.

In respect to the degree of fusion proteins purification in the RPBLAs fractions (F56 and F65), it was estimated that RX3-EGF protein represents approximately 80 percent of the proteins detected in the PBLS-containing fractions. This result indicates that, using a RPBLAs isolation procedure, one can achieve an important enrichment of fusion proteins in only one step of purification.

### EXAMPLE B

### Recombinant Proteins Recovery in RPBLAs Isolated from Dry Plant Tissues

An important point in molecular farming is the presence of an easy means to store plant biomass. In this context, drying can provide a convenient method to lessen storage volume and preserve the product. Nevertheless, drying frequently promotes the degradation of the proteins of interest. The use of desiccated plants to isolate RPBLAs containing recombinant proteins would be of great interest for industrial purposes.

Transformed tobacco leaves accumulating RX3-EGF and RX3-T20 fusion proteins as described above were dried as also discussed above. After 5 months of dry storage, the stability of recombinant proteins was analyzed. Protein extracts from equivalent amounts of wet (fresh) (W, lane 1) and dry (D, lane 2) leaf tissue were analyzed by immunoblot (FIG. 2D). As can be seen in the figure, RX3-EGF protein was stable in desiccated transformed plants, the amount recovered in wet and dry plants being similar (compare lanes W and D).

The distribution in step density gradients of fusion proteins (RX3EGF and RX3T20) from homogenates of dried leaves was analyzed by immunoblot (FIG. 2 D, lanes 3-7). Interestingly, both fusion proteins were mainly recovered in dense structures showing densities higher than 1.1868 g/cm³ (F42 fraction) and 1.2632 g/cm³ (F56 fraction).

Thus, recombinant proteins can be purified from dried tissues via isolation of RPBLAs thereby illustrating that transgenic plant collection and recombinant protein extraction and purification can be independent in time. In keeping with these results, gamma-zein fusion proteins were also accumulated in RPBLAs in rice seeds.

### EXAMPLE C

### Recombinant Protein Recovery by Isolation of RPBLAs from Transiently Transformed Tobacco Plantlets

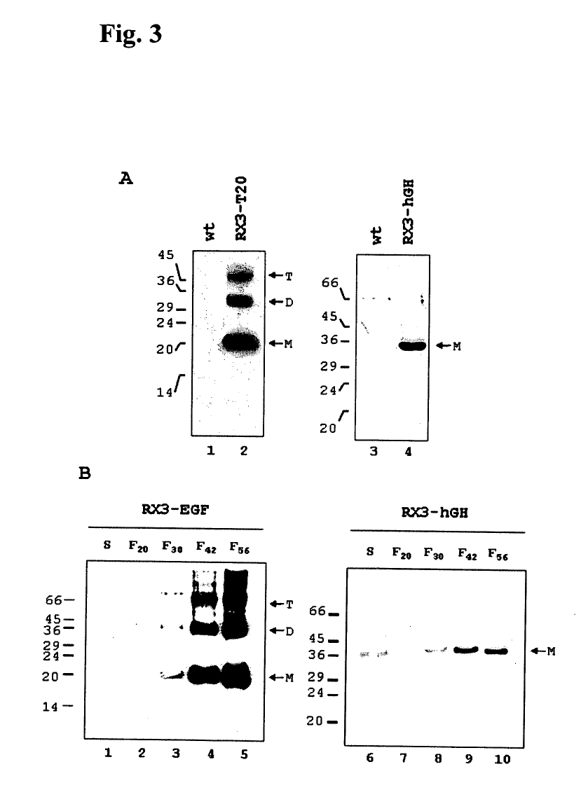

The transient expression systems can be a convenient tool to test the accumulation behavior of recombinant proteins in a short period of time. Thus, the recombinant proteins RX3-EGF and RX3-T20 were also expressed and accumulated in transiently transformed tobacco plantlets via agroinfiltration. The protein extracts from transformed plantlets analyzed by immunoblot (FIG. 3A) show the characteristic complex electrophoretic pattern observed from stably-transformed plants (compare FIGS. 3A, lane 4 and 2A, lane4), indicating that the fusion proteins assemble correctly using this method of transformation.

The expression of a higher molecular weight fusion protein, RX3-hGH was also analyzed in transiently transformed tobacco (FIG. 3A, lane 4). After sub-cellular fractionation on density gradients both, RX3-T20 and RX3-hGH, fusion proteins were recovered in dense fractions corresponding to RPBLAs fractions (FIG. 3B, lanes 4, 5 and 9, 10) showing densities higher than 1.1868 g/cm³ (F42) and 1.2632 g/cm³ (F56). Transient expression can thus be used to test, in a short period of time, the particular density properties of PBs containing a desired recombinant protein.

### EXAMPLE D

### Recovery of Recombinant Proteins by Low and Medium Speed Centrifugation

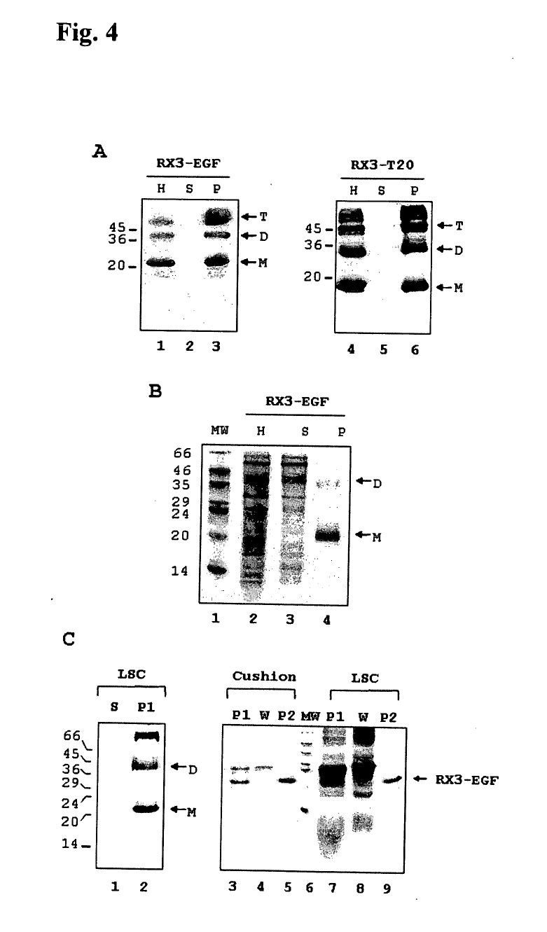

To simplify the procedure used to purify recombinant proteins via dense recombinant protein body-like assemblies, two additional alternative methods were performed: i) clarified homogenates were centrifuged through only one dense sucrose cushion (FIG. 4 A, B) and ii) clarified homogenates were simply centrifuged at low speed centrifugation (i.e. 1000-2500×g for 10 minutes).

In agreement with the previously described results, both RX3-EGF and RX3-T20 proteins were recovered in high yields (more than 90%) in the pellets obtained after centrifugation through 1.1868 g/cm3 sucrose cushions (FIG. 4A, lanes 4 and 6). In addition, the purification of RX3-EGF protein was very high as can be seen in the silver stained gel of FIG. 4B, where contaminant tobacco endogenous proteins were barely detected in the corresponding pellet (lane 4).

The principal advantage of this method as compared to step density gradients lies in its easy scalability for industrial production of recombinant proteins. It should be noted that the cushion density as well other properties such as its viscosity and osmolarity can be adjusted in each case in order to optimize recovery and purification of the recombinant proteins.

In addition, low speed centrifugation (LSC) was also assayed to concentrate and purify fusion protein-containing protein body-like structures (FIG. 4C, LSC). The results indicated that, after 1000×g for 10 minutes, practically all the RX3-EGF fusion protein was recovered in the pellet (FIG. 4C lane 2). But the staining of the proteins contained in this pellet revealed that the fusion protein was not highly purified as compared with that obtained after centrifugation through 1.1868 g/cm3 sucrose cushion (FIG. 4C, compare lanes 3 and 7).

Thereafter, the first pellet obtained by low speed centrifugation was washed by using a buffer containing 5% Triton X-100. After washing, the sample was centrifuged at 12,000×g for 5 minutes and, interestingly, the bulk of contaminating proteins present in the P1 pellet were eliminated after washing and centrifugation and the new pellet (P2, FIG. 4C, lane 9) contained a highly enriched RX3-EGF protein. It is noted that the amount as well the pattern of proteins in lane 9 is similar to that obtained after washing the pellet obtained after centrifugation through the sucrose cushion in the Triton X-100-containing buffer (FIG. 4C, lane 5). The low speed centrifugation alternative is based on the high density of the structures containing fusion proteins and centrifugation conditions can be optimized for every target before to scale up.

### EXAMPLE E

### Recombinant Protein Recovery by Isolation of RPBLAs from Transfected Animal Cells

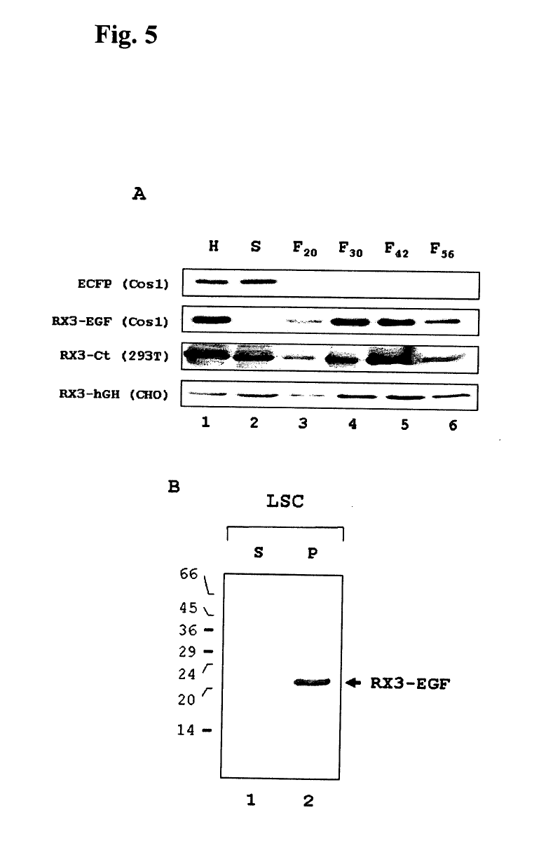

Studies were undertaken to determine whether storage protein-derived fusion proteins also induced the formation of dense recombinant PB-like assemblies in transfected animal cells. The sub-cellular distribution of organelles from homogenized transfected mammal cells was analyzed by using step density gradients. Three different cell culture types, 293T (from human), Cos1 (from monkey) and CHO (from hamster), were transfected by using the cDNA coding for three different fusion proteins, RX3-Ct, RX3-EGF and RX3-hGH. Cos1 cells transfected with pECFP-N1 (Clontech) were used as control. The gradient fractions were collected as described previously and analyzed by immunoblot (FIG. 5A).

The recombinant RX3-derived proteins expressed in transfected cells, were detected by using the gamma-zein antiserum. Detection of the control ECGP in the different collected fractions was made by using an anti-GFP antiserum raised in rabbits.

As expected, the soluble ECGP protein was recovered in the supernatant fraction (S, FIG. 5A, lane 2) and no traces of this protein were detected in the interphase and pellet fractions where particulate cell fractions are sedimented. In contrast, RX3CT, RX3EGF and RX3hGH were mainly present in the dense fractions F30, F42 and F56 (FIG. 5A), indicating that gamma-zein derived fusion proteins could be recovered from these dense fractions (densities from 1.1270 to 1.2632 g/cm³). These results agree with those obtained by immunocytochemistry, where fusion proteins were located in the ER and in recombinant protein body-like assemblies of about 1 to about 1.4 microns in diameter.

A significant amount of recombinant protein was recovered in the soluble fraction of the gradients from RX3-Ct- and RX3-hGH-transfected cells. This was probably due to an excess of cell rupture during homogenization that permitted the solubilization of the yet unassembled fusion proteins contained in the ER.

Low speed centrifugation (LSC, FIG. 5B) was also assayed by using homogenates from RX3-EGF expressing CHO cells. As can be seen in FIG. 5B, the bulk of fusion protein was recovered in the 2500×g pellet (lane 2), confirming that fusion proteins accumulate in dense protein body-like structures in animal cells that can be recovered by density based methods.

### EXAMPLE F

### Recovery of Recombinant Proteins from Transformed Yeast by Density Gradients

The formation of dense structures containing fusion proteins in transformed yeast was also analyzed by step density gradients. The cDNAs coding for RX3-EGF and RX3-hGH fusion proteins were introduced via yeast transformation vectors in Sacharomyces cerevisiae using standard procedures. The disruption method applied to isolate organelles was based in the gentle lysis of spheroplasts as described in methods. The lysates were loaded on a step sucrose gradient and centrifuged as described for mammal cells or tobacco leaf homogenates. Fractions were analyzed by SDS-PAGE and immunoblot.

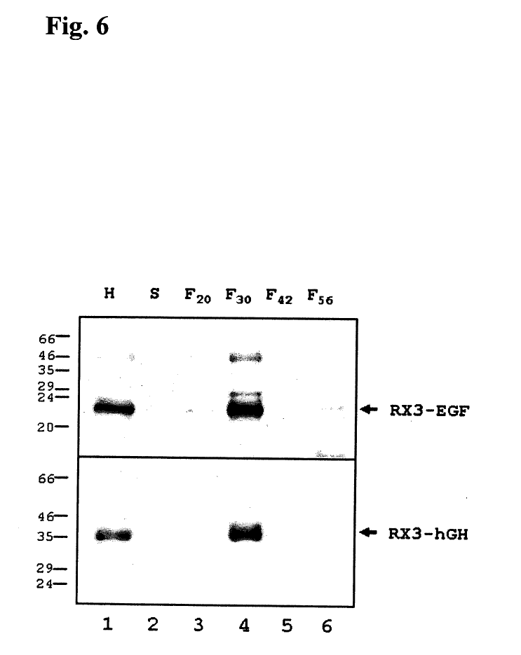

The fractionation results are shown in FIG. 6 where it can be seen that the most of both, RX3-EGF and RX3-hGH, proteins were located in the interphase F30 (FIG. 6, lane 4) of the gradients. This fraction contains sub-cellular structures with densities between 1.1270 and 1.1868 g/cm3. No significant amounts of fusion proteins were detected in the supernatant and the F20 fraction, indicating that the fusion proteins are assembled in the yeast cells. It is possible that the reduced size of the yeast cells (up to 3 microns) only permits the formation of small RPBLAs, which are less dense than those observed in plants and animal cells. In any case, they can be isolated and purified from most other cellular proteins by centrifugation.

### EXAMPLE G

### Recovery of Fusion Proteins from Different Storage Protein Domains by Density Gradients

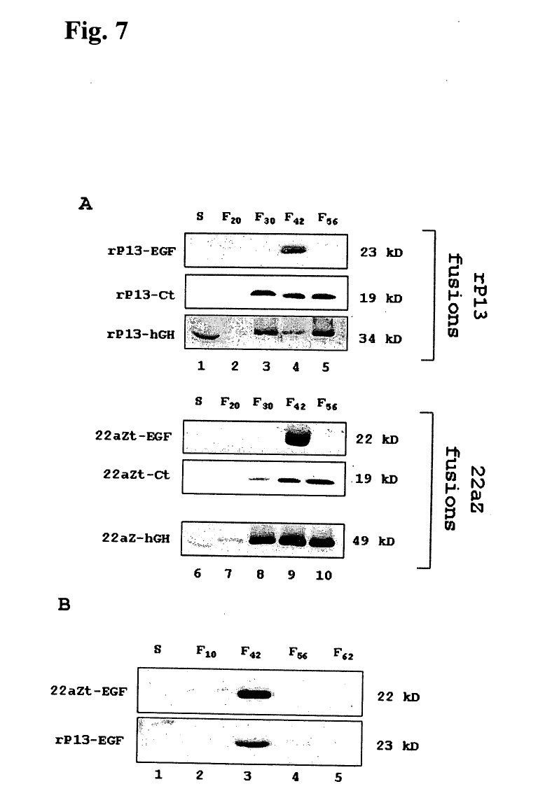

The purification of gamma-zein derived fusion proteins through their density properties is extensible to fusion proteins derived from other storage proteins. Here we show how fusion proteins derived from the rice prolamin of 13 kD (rP13) and from 22 kD alfa-zein (22aZt) accumulated in dense fractions corresponding to RPBLAs on step sucrose gradients (FIG. 7). The selection of the rice prolamin of 13 kD (rP13) and the 22 kD alfa-zein [in the full lengh version (22aZ) or the N-terminal domain (22aZt)] was based in the lack of homology between them, and with regard to the RX3 domain. Unexpectedly, both storage proteins produced high dense RPBLAs, which were recovered in the denser interfaces when submitted to step density gradients (FIG. 7A).

The calcitonin sequence was fused in frame with rP13 and 22aZt sequences under the CaMV35S promoter and introduced in Agrobacterium tumefaciens. Tobacco plantlets were transiently transformed and leaf homogenates were submitted to step density gradients. The collected fractions were analyzed by SDS-PAGE and immunoblot using an anti-calcitonin antiserum raised in rabbit.

As can be seen in FIG. 7A (lanes 4-5, and 9-10), the larger amounts of both, rP13Ct and 22aZtCt fusion proteins were located in the F42 and F56 fractions, indicating the presence of recombinant PB-like assemblies with densities higher than 1.1868 g/cm3 that can be isolated for fusion protein purification. The results also indicate that the density of recombinant PB-like assemblies containing fusion proteins can vary in function of the storage protein included in the fusion.

Agroinfiltration studies were performed with the hGH fused to the rice prolamin (rP13-hGH) and the full length alpha zein (22aZ-hGH). Once again, the majority of the RPBLAs were observed in the F42 and F56 fractions 7A (lane 4-5 and lanes 9-10), but interestingly this time some non-assembled fusion protein was also detected in the supernatant and in the low density interfaces (lanes 1-2 and 6-7). This effect could be explained by a partial solubilizing effect of the hGH to the rP13 and the 22aZ.

Transgenic tobacco plants expressing the fusion proteins rP13-EGF and the 22aZ-EGF were produced by Agrobacterium tumefasciens transformation. The best expressers where determined by immunoblot using an antibody against the EGF, and those cell lines were used in a comparative analysis with tobacco plantlets agroinfiltrated with the same constructs. As can be seen in FIG. 7 (A, lanes 4 and 9; B lane 3) in all the cases, the RPBLAs where recovered in unique interface (F12), suggesting that the RPBLAs are very dense and homogeneous.

Taking all these results together, it is clear that prolamins are able to induce high density RPBLAs, even when they are fused to other proteins. That is an unexpected result, mainly when almost no homology is observed between them. Moreover, there are some data suggesting that the prolamins interact to stabilize the protein bodies, and that some of them are not stable when expressed in vegetative tissue alone, as for instance alpha-zein (Coleman et al., 1996 Plant Cell 8:2335-2345)

### EXAMPLE H

### Extraction of Recombinant Proteins from Isolated PBLS

It has been demonstrated that the isolation of dense recombinant PB-like assemblies is an advantageous method to recover recombinant proteins with high yield and high purification level from transgenic organisms. Here it is shown that these recombinant proteins can be extracted from the storage organelles. Although the recombinant PBLAs can be directly used for some applications (i.e. oral vaccine preparation), in some other cases, the disposal of purified recombinant proteins could be necessary.

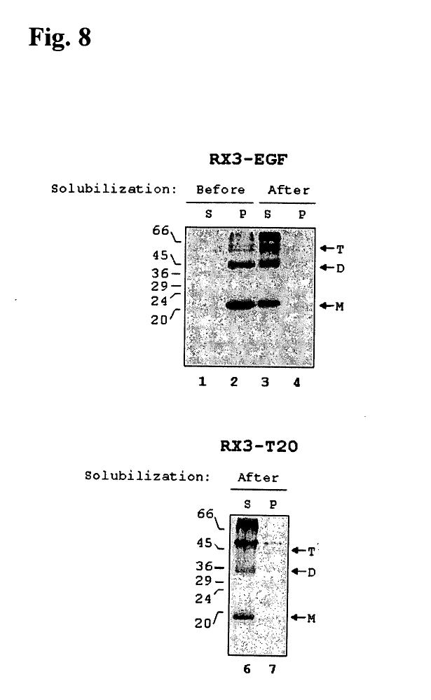

After an overnight (about 18 hours) incubation of PB fractions at 37° C. in a buffer containing reducing agents (SB buffer that contained sodium borate 12.5 mM pH 8, 0.1% SDS and 2% 2-mercaptoethanol; treatment), RX3-EGF and RX3-T20 proteins were solubilized. As can be seen in FIG. 8 lanes 1-4 for RX3-EGF and lanes 6-7 for RX3-T20, both extracted fusion proteins were recovered in their soluble forms (S). Afterwards, as a function of their application, the extracted proteins can be submitted to further purification or used as partially purified extracts.

Each of the patents and articles cited herein is incorporated by reference. The use of the article “a” or “an” is intended to include one or more.

The foregoing description and the examples are intended as illustrative and are not to be taken as limiting. Still other variations within the spirit and scope of this invention are possible and will readily present themselves to those skilled in the art.

What is claimed:

1. A method for purifying recombinant fusion protein expressed as recombinant protein body-like assemblies in host cells that comprises the steps of:
(a) providing an aqueous homogenate of transformed host cells that express a fusion protein as recombinant protein body-like assemblies (RPBLAs), said RPBLAs having a predetermined density;
(b) forming regions of different density in the homogenate to provide a region that contains a relatively enhanced concentration of the RPBLAs and a region that contains a relatively depleted concentration of the RPBLAs; and
(c) separating the RPBLAs-depleted region from the region of relatively enhanced concentration of RPBLAs, thereby purifying said fusion protein.

2. The method according to claim 1 wherein said predetermined density of the RPBLAs is greater than that of substantially all of the endogenous host cell proteins present in the homogenate.

3. The method according to claim 1 wherein said fusion protein contains two sequences linked together in which one sequence is a protein body-inducing sequence and the other is the sequence of a product of interest.

4. The method according to claim 1 wherein said regions of different density in the homogenate are provided by centrifuging the homogenate in the presence of a differential density-providing solute.

5. A method for purifying recombinant fusion protein expressed as recombinant protein body-like assemblies (RPBLAs) in host cells that comprises the steps of:
(a) providing a clarified aqueous homogenate of transformed host cells that express a fusion protein as RPBLAs, said RPBLAs having a predetermined density, wherein said fusion protein contains two sequences linked together in which one sequence is a protein body-inducing sequence and the other is the sequence of a product of interest;
(b) centrifuging the homogenate in the presence of a differential density-providing solute to form regions of different density in the homogenate and provide a region that contains a relatively enhanced concentration of the RPBLAs and a region that contains a relatively depleted concentration of the RPBLAs; and
(c) separating the RPBLAs-depleted region from the region of relatively enhanced concentration of RPBLAs, thereby purifying said fusion protein.

6. The method according to claim 5 wherein said regions of different density are provided by a density gradient.

7. The method according to claim 5 wherein said regions of different density are provided by a density cushion having a density that is less than that of said RPBLAs.

8. The method according to claim 5 wherein said regions of different density are provided by the supernatant and the pellet obtained after low speed centrifugation of the homogenate.

9. The method according to claim 5 wherein said host cells are higher plant cells.

10. The method according to claim 5 wherein said host cells are fungi cells.

11. The method according to claim 5 wherein said host cells are particularly yeast cells.

12. The method according to claim 5 wherein said host cells are algal cells.

13. The method according to claim 5 wherein said host cells are animal cells.

14. The method according to claim 5 wherein said host cells are particularly mammalian cells.

15. The method according to claim 5 wherein said fusion protein further includes a linker sequence between the protein body-inducing sequence and the sequence of the product of interest.

16. The method according to claim 5 wherein the protein body-inducing sequence comprises a prolamin or a modified prolamin.

17. The method according to claim 5 wherein the RPBLA has a density of about 1.1 to about 1.35 g/ml.

18. A method for purifying recombinant fusion protein expressed as recombinant protein body-like assemblies (RPBLAs) in host cells that comprises the steps of:
(a) providing a clarified aqueous homogenate of transformed host plant cells that express a fusion protein as RPBLAs, said RPBLAs having a predetermined density of about 1.1 to about 1.35 g/ml, wherein said fusion protein contains two sequences linked together in which one sequence is a protein body-inducing sequence that is a prolamin or modified prolamin and the other is the sequence of a product of interest;
(b) centrifuging the homogenate in the presence of a differential density-providing solute to form a homogenate-solute admixture, said homogenate-solute admixture having regions of different density by formation of a density gradient or a density cushion having a density that is less than that of said RPBLAs, and providing a region that contains a relatively enhanced concentration of the RPBLAs and a region that contains a relatively depleted concentration of the RPBLAs; and
(c) separating the RPBLAs-depleted region from the region of relatively enhanced concentration of RPBLAs, thereby purifying said fusion protein.

19. The method according to claim 18 wherein the homogenate is prepared from fresh biomass.

20. The method according to claim 18 wherein the homogenate is prepared particularly from fresh plant cells.

21. The method according to claim 18 wherein the homogenate is prepared from dried biomass.

22. The method according to claim 18 wherein the homogenate is prepared from dried plant cells.

23. The method according to claim 18 wherein the protein body-inducing sequence is a prolamin sequence.

24. The method according to claim 23 wherein the prolamin sequence is gamma-zein, alpha-zein or rice prolamin.

25. The method according to claim 18 including the further step of recovering the RPBLAs.
